(19) United States
(12) Patent Application Publication
(10) Pub. No.: US 2010/0003331 A1
(43) Pub. Date: Jan. 7, 2010

(54) SUSTAINED RELEASE DOSAGE FORMS OF ZIPRASIDONE

(75) Inventors: Imran Ahmed, East Lyme, CT (US); Leah Elizabeth Appel, Bend, OR (US); Walter Christian Babcock, Bend, OR (US); Dwayne Thomas Friesen, Bend, OR (US); Scott Herbig, East Lyme, CT (US); David Keith Lyon, Bend, OR (US); Sheri L. Shamblin, North Stonington, CT (US); Ravi Mysore Shanker, Groton, CT (US); Daniel Tod Smithey, Bend, OR (US); Steven C. Sutton, Niantic, CT (US); Avinash Govind Thombre, East Lyme, CT (US); Kenneth C. Waterman, Waterford, CT (US); Jaymin C. Shah, Waterford, CT (US); Parag Suresh Shah, Groton, CT (US); Peter Wisniecki, Coventry, CT (US); Dawn Renee Wagner, Groton, CT (US)

Correspondence Address: PFIZER INC;Mary J Hosley, 150 EAST 42ND STREET, MS: 150/02/E112, NEW YORK, NY, 10017-5612, US

(73) Assignee: Pfizer Inc., New York, NY (US)

(21) Appl. No.: 12/261,678

(22) Filed: Sep. 3, 2009

Related U.S. Application Data
(63) Continuation of application No. 10/931,143, filed on Aug. 31, 2004.
(60) Provisional application No. 60/499,484, filed on Sep. 2, 2003.

(51) Int. Cl. A61K 9/14; A61K 31/501

(52) U.S. Cl. 424/489; 514/254.02; 977/906

(57) ABSTRACT

A sustained release solid oral dosage form for treatment of a psychotic disorder, for example schizophrenia, in a mammal is provided, which oral dosage form comprises ziprasidone in an amount effective in treating said psychotic disorder and a pharmaceutically acceptable carrier.

This application claims priority under 35 USC 119 of U.S. Provisional 60/499,484, filed Sep. 2, 2003.

### BACKGROUND

The invention relates to sustained release dosage forms comprising ziprasidone.

Ziprasidone is an atypical antipsychotic medication currently marketed in the United States as GEODON®, in both an immediate-release (IR) oral capsule formulation for the acute and long-term treatment of schizophrenia and an IR intramuscular (IM) formulation for acute control of agitation in patients with schizophrenia. The IR oral capsule is typically taken twice per day. The IR oral capsule is available as 20, 40, 60, and 80 mgA capsules. (By “mgA” is meant the amount of active ziprasidone—that is, ziprasidone freebase in mg.) The initial dose is typically 20 mgA twice a day taken with food. The dose is then adjusted based on the patient's response.

It is desired to provide an oral sustained release ziprasidone dosage form. Such a dosage form should provide efficacious blood levels of ziprasidone over a longer period of time than the IR oral capsule, but ideally would not provide maximum blood levels that are higher than those provided by an IR oral capsule containing the same amount of ziprasidone. Such a dosage form may increase patient compliance and maximize patient and physician acceptance, such as by reducing side effects. Such a dosage form may also provide a safety and tolerability profile as good as or better than the IR oral capsule regimen due to relatively lower blood levels of ziprasidone compared with the IR oral capsule at the same dose.

To achieve efficacious blood levels over long periods of time, the sustained release dosage form should release ziprasidone to the gastrointestinal tract in a manner that allows ziprasidone to be absorbed for a sustained length of time. However, formulating ziprasidone into a sustained release dosage form presents a number of problems. While ziprasidone has relatively good solubility at gastric pH, it has relatively poor solubility at intestinal pH. The free base form of ziprasidone has a solubility of about 0.2 μg/ml at a pH of about 6.5. Such low solubility at intestinal pH inhibits absorption of ziprasidone in the intestines. In addition, if ziprasidone becomes supersaturated in an aqueous solution (that is, dissolved at a concentration that is greater than the equilibrium solubility of the drug at intestinal pH, such as occurs when moving from a low-pH gastric environment to a higher pH intestinal environment), it has a tendency to rapidly precipitate as the crystalline free base form of the drug, thus rapidly reducing the concentration of dissolved ziprasidone to the solubility of the free base crystalline (lowest energy form) of ziprasidone.

Curatolo et al., U.S. Pat. No. 6,548,555 B1 disclose mixtures of basic drugs and precipitation inhibiting polymers such as hydroxypropyl methyl cellulose acetate succinate (HPMCAS). Curatolo et al. teach that the drug will dissolve in the stomach, and the precipitation-inhibiting polymer will maintain high dissolved drug concentration as the dissolved drug enters the intestines.

Curatolo et al., US Publication No. 2002/0006443 A1 and Curatolo et al., US Publication No. 2003/0072801 A1 disclose physical mixtures of solubility-improved forms of low-solubility drugs combined with polymers to provide enhancement of the aqueous concentration of dissolved drug. In particular, various solubility-improved forms of ziprasidone mixed with polymers such as hydroxypropyl methyl cellulose acetate succinate are disclosed.

WO 01/47500 discloses an osmotic controlled release dosage form. The application discloses in Example 10 an osmotic dosage form containing 20 mgA of ziprasidone in the form of a solid amorphous dispersion of the drug in the polymer hydroxypropylmethyl cellulose acetate succinate.

It is desired to provide an oral dosage form to allow sustained release of ziprasidone that delivers a pharmaceutically effective amount of ziprasidone to a patient in need thereof.

### SUMMARY

The present invention provides a sustained release (SR) solid oral dosage form for treatment of a psychotic disorder, for example schizophrenia, in a mammal, which oral dosage form comprises ziprasidone in an amount effective in treating said psychotic disorder and a pharmaceutically acceptable carrier.

Accordingly, the present invention provides a solid oral dosage form for treatment of a psychotic disorder, for example schizophrenia, in a mammal which oral dosage form comprises ziprasidone in an amount effective in treating said psychotic disorder and a pharmaceutically acceptable carrier, wherein the effective amount of ziprasidone is released over a sustained period of time.

In one embodiment, the oral dosage form is a tablet. In another embodiment, the oral dosage form is a capsule.

In another embodiment, the sustained period of time is at least about 24 hours. In other embodiments, the sustained period of time ranges from about 4 hours to about 24 hours. The sustained period of time may be at least about 4 hours, at least about 6 hours, at least about 8 hours, at least about 10 hours, at least about 12 hours, or at least about 16 hours. In another embodiment, the sustained period of time is about 24 hours. Using the phrase “at least about 6 hours” as an example, the phrase “at least about”, as used in such context, means in one embodiment that substantially all (e.g. about 80 wt % or more), of the ziprasidone in the dosage form is released from the dosage form following administration over a period of time of about 6 hours, with no more than about 20 wt % being released after 6 hours. In another embodiment, it means that substantially all (e.g., about 80 wt % or more) of the ziprasidone is released from the dosage form following administration over a period of time longer than about 6 hours.

In another embodiment, the oral dosage form comprises more than one layer, for example 2 or 3 layers. In a preferred embodiment, the oral dosage form comprises a bi-layer core, comprising an active layer and a sweller layer. The core may be coated. The oral dosage form comprising multiple layers may, in one embodiment, comprise one or more holes on the surface of the coating on the active layer side.

In one aspect, a sustained release oral dosage form comprises a pharmaceutically effective amount of ziprasidone and sustained release means for releasing at least a portion of the ziprasidone, wherein following administration to achieve steady state, the dosage form provides a steady state minimum blood ziprasidone concentration (Cₘᵢₙ) of at least 20 ng/ml, and a steady state maximum blood ziprasidone concentration (Cₘₐₓ) of less than 330 ng/ml.

By blood ziprasidone concentration is meant concentration of ziprasidone in blood, in serum, or in plasma.

In one preferred embodiment the steady state ratio of Cₘₐₓ to Cₘᵢₙ is less than about 2.6 when dosed twice per day. In another preferred embodiment, the ratio of Cₘₐₓ to Cₘᵢₙ is less than about 12 when dosed once per day.

In a second aspect, a pharmaceutical dosage form comprises a pharmaceutically effective amount of ziprasidone, the dosage form releasing no greater than about 90 wt % of the total amount of ziprasidone from the dosage form during the first 2 hours after administration to a use environment. The dosage form contains at least 30 mgA of ziprasidone.

As used herein, a “use environment” can be either the in vivo environment, such as the GI tract of an animal, particularly a human, or the in vitro environment of a test solution, such as phosphate buffered saline (PBS) solution, Model Fasted Duodenal (MFD) solution, or a simulated intestinal buffer solution.

In a third embodiment, a sustained release dosage form comprises a pharmaceutically effective amount of ziprasidone and sustained release means for releasing at least a portion of the ziprasidone. The ziprasidone contained in the sustained release portion is at least one of (i) crystalline drug and (ii) drug combined with cyclodextrin.

In another aspect, the invention provides a method for administering ziprasidone. The method comprises administering a sustained release dosage form, that when dosed either once or twice per day to a human in the fed state, provides a minimum steady state blood ziprasidone concentration (Cₘᵢₙ) of at least about 20 ng/ml, and a maximum steady state blood ziprasidone concentration (Cₘₐₓ) of less than about 330 ng/ml.

In one preferred embodiment of the method, the steady state ratio of Cₘₐₓ to Cₘᵢₙ is no greater than about 2.6 when dosed twice per day. In another preferred embodiment, the ratio of Cₘₐₓ to Cₘᵢₙ is no greater than about 12 when dosed once per day.

“Sustained release” means that the dosage form releases no greater than about 90 wt % of the ziprasidone in the dosage form during the first two hours after administration to a use environment. Thus the dosage form may release ziprasidone gradually and continuously over a release period, may release ziprasidone in a pulsatile or delayed manner, or may release ziprasidone in a combination of release profiles, such as an immediate release burst followed by either a delayed burst or by a gradual and continuous release.

“Administration” to a use environment means, where the in vivo use environment is the GI tract, delivery by ingestion or swallowing or other such means to deliver the dosage form. Where the use environment is in vitro, “administration” refers to placement or delivery of the dosage form to the in vitro test medium.

A sustained release dosage form may provide a number of advantages. Without wishing to be bound by theory, it is believed that ziprasidone efficacy is related to occupancy of the D2 receptor. Occupancy in turn is a function of the concentration of ziprasidone in the brain, which is related to the concentration of ziprasidone in the blood, with occupancy increasing substantially as the concentration of ziprasidone in the blood increases. D2 occupancy is approximately 50% when the blood ziprasidone concentration is 16 ng/ml, approximately 65% when the blood ziprasidone concentration is 30 ng/ml, and approximately 75% when the blood ziprasidone concentration is 50 ng/ml. Accordingly it is preferred that the dosage form provide a minimum steady state blood ziprasidone concentration of at least about 20 ng/ml for efficacy, more preferably at least about 30 ng/ml, and even more preferably at least about 50 ng/ml. A sustained release dosage form may improve efficacy by maintaining the blood level of ziprasidone at high enough concentrations to provide greater D2 occupancy for a longer period of time than the IR oral capsule. This may be achieved because the sustained release dosage form may permit dosing of greater amounts of ziprasidone relative to the IR oral capsule, or may be due to absorption of ziprasidone over a longer period of time relative to the IR oral capsule, or both. The sustained release dosage form may also minimize the fluctuation in blood levels of ziprasidone, thereby yielding a more uniform response.

A sustained release dosage form may also provide lower maximum blood levels of ziprasidone relative to the IR oral capsule for a given dose, thus potentially reducing or mitigating adverse events or side effects. Alternatively, a higher dose sustained release dosage form of ziprasidone may be administered, which would result in greater efficacy compared to a lower dose IR oral capsule, and fewer adverse events or side effects relative to a higher dose IR oral capsule.

For those sustained released formulations which provide for once a day administration, the sustained release dosage forms may provide greater convenience and compliance arising out of once daily dosing. This is particularly important because the absorption of ziprasidone is increased up to two-fold in the presence of food and so it is recommended that ziprasidone be administered with food. Compliance to “take with food” is likely to be better when the dosing frequency is once or twice a day compared to several times a day.

The foregoing and other objectives, features, and advantages of the invention will be more readily understood upon consideration of the following detailed description of the invention.

### BRIEF DESCRIPTION OF THE DRAWINGS

FIG. 1 shows ziprasidone concentration in the blood (plasma) versus time for a model dosage form based on the modeling results of Ex. 4.

FIG. 2 shows ziprasidone concentration in the blood (plasma) versus time for another model dosage form based on the modeling results of Ex. 4.

### DETAILED DESCRIPTION OF THE INVENTION

Ziprasidone is 5-[2-[4-(1,2-benzisothiazol-3-yl)-1-piperazinyl]ethyl]-6-chloro-1,2-dihydro-2H-indol-2-one, a known compound having the structure:

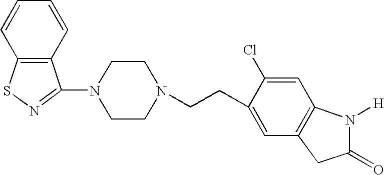

Ziprasidone is disclosed in U.S. Pat. Nos. 4,831,031 and 5,312,925, both of which are herein incorporated by reference in their entirety. Ziprasidone has utility as a neuroleptic, and is thus useful, inter alia, as an antipsychotic. Ziprasidone is typically administered in a daily dose of from about 40 mgA to about 160 mgA, depending on patient need. By “daily dose” is meant the total amount of ziprasidone administered to a patient in one day.

The term “ziprasidone” should be understood to include any pharmaceutically acceptable form of the compound. By “pharmaceutically acceptable form” is meant any pharmaceutically acceptable derivative or variation, including stereoisomers, stereoisomer mixtures, enantiomers, solvates, hydrates, isomorphs, polymorphs, pseudomorphs, neutral forms, acid addition salt forms, and prodrugs. The pharmaceutically acceptable acid addition salts of ziprasidone are prepared in a conventional manner by treating a solution or suspension of the free base with about one chemical equivalent of a pharmaceutically acceptable acid. Conventional concentration and recrystallization techniques are employed in isolating the salts. Illustrative of suitable acids are acetic, lactic, succinic, maleic, tartaric, citric, gluconic, ascorbic, mesylic, tosylic, benzoic, cinnamic, fumaric, sulfuric, phosphoric, hydrochloric, hydrobromic, hydroiodic, sulfamic, sulfonic such as methanesulfonic, benzenesulfonic, and related acids. Preferred forms of ziprasidone include the free base, ziprasidone hydrochloride monohydrate, ziprasidone mesylate trihydrate, and ziprasidone tosylate.

The oral sustained-release dosage forms of the present invention contain a sufficient amount of ziprasidone so as to be pharmaceutically effective. The typical daily dose for ziprasidone ranges from 40 mgA to 240 mgA ziprasidone. One or multiple sustained release dosage forms may be administered simultaneously to achieve the desired dose. In preferred embodiments, the sustained release dosage form contains at least about 40 mgA to about 160 mgA ziprasidone.

Since the dosage forms may contain a relatively large amount of ziprasidone, it is desired, to accommodate the high drug loading, that ziprasidone constitutes a significant fraction of the dosage form. This allows the dosage form to be kept at a size that is convenient for oral administration (e.g., preferably less than 1,000 mg, and more preferably less than 800 mg). Preferably, ziprasidone constitutes at least about 5 wt % of the dosage form. Ziprasidone may constitute even greater amounts of the dosage form, such as at least about 10 wt %, or even at least about 15 wt % of the dosage form.

Ziprasidone may be present in crystalline or amorphous form. Because ziprasidone has a tendency to rapidly crystallize, the crystalline form is preferred from the standpoint of stability of the drug in the dosage form. When present as amorphous drug, ziprasidone is preferably present in a stable form. A preferred amorphous form is a co-lyophile of ziprasidone and cyclodextrin.

The ziprasidone in the sustained-release dosage form may optionally be in a solubility-improved form. By a “solubility-improved form” is meant a form of ziprasidone that is capable of providing concentration-enhancement as described in more detail below. Solubility-improved forms of ziprasidone are described in more detail below. As discussed herein, a solubility-improved form is preferred for those embodiments in which it is desired to achieve absorption of ziprasidone in the distal small intestine or in the colon, and for those embodiments in which it is desired to provide once a day administration.

In one embodiment, the solubility-improved form of ziprasidone is a high solubility salt form. It is known that some low-solubility drugs may be formulated in highly soluble salt forms that provide temporary improvements in the concentration of the drug in a use environment relative to another salt form of the drug. An example of such a salt form for ziprasidone is the mesylate salt, which has an aqueous solubility of about 900 μg/mL at pH of 2.5. The solubility of several high-solubility salt forms of ziprasidone are given in the following table:

| | Aqueous Solubility | pH of Saturated |
|---|---|---|
| Salt Form | (μg/mL) | Solution |
| Free base | 0.2 | 9.8 |
| Hydrochloride | 12 | 4.3 |
| Mesylate | 900 | 2.5 |
| Citrate | 86 | 4.1 |
| Phosphate | 37 | 2.3 |
| Tosylate | 64 | 6.0 |
| Maleate | 118 | 4.3 |
| Succinate | 187 | 3.4 |
| Salicylate | 58 | 5.5 |
| Fumarate | 2000 | 2.5 |

Preferred high-solubility salt forms of ziprasidone include the hydrochloride, mesylate, tosylate, phosphate and salicylate.

In another embodiment, the solubility-improved form comprises ziprasidone having a volume weighted mean particle size of less than about 10 μm and preferably less than about 5 μm. Standard crystalline ziprasidone HCl is typically in block or needle habits. The size of such crystals is commonly 30 μm long and 4 μm wide, but there is a wide range observable. When these crystals are analyzed by a Malvern Mastersizer and studied as a wet slurry, the volume-weighted mean diameter is about 10 μm. Reducing the particle size of ziprasidone may improve its dissolution rate, thus providing at least temporarily enhanced concentrations of dissolved ziprasidone in an aqueous use environment relative to the concentration achieved with larger crystal sizes. Such small particles may be achieved by conventional grinding and milling techniques. In one preferred process, the ziprasidone is jet milled. Jet-milled ziprasidone may have a volume weighted mean diameter of less than about 5 microns, and preferably less than about 3 microns.

In another embodiment, the ziprasidone may be in the form of nanoparticles. The term “nanoparticle” refers to ziprasidone in the form of particles generally having an effective average crystal size of less than about 500 nm, more preferably less than about 250 nm and even more preferably less than about 100 nm. Examples of such nanoparticles are further described in U.S. Pat. No. 5,145,684, herein incorporated by reference. The nanoparticles of the drug can be prepared using any known method for preparing nanoparticles. One method comprises suspending ziprasidone in a liquid dispersion medium and applying mechanical means in the presence of grinding media to reduce the particle size of the drug substance to the effective average particle size. The particles can be reduced in size in the presence of a surface modifier. Alternatively, the particles can be contacted with a surface modifier after attrition. Other alternative methods for forming nanoparticles are described in U.S. Pat. No. 5,560,932, and U.S. Pat. No. 5,874,029, both incorporated by reference in their entirety.

Another solubility-improved form of ziprasidone comprises ziprasidone combined with a cyclodextrin (as an inclusion complex or as a physical mixture). As used herein, the term “cyclodextrin” refers to all forms and derivatives of cyclodextrin. Particular examples of cyclodextrin include α-cyclodextrin, β-cyclodextrin, and γ-cyclodextrin. Exemplary derivatives of cyclodextrin include mono- or polyalkylated β-cyclodextrin, mono- or polyhydroxyalkylated β-cyclodextrin, such as hydroxypropyl β-cyclodextrin (hydroxypropylcyclodextrin), mono, tetra or hepta-substituted β-cyclodextrin, and sulfoalkyl ether cyclodextrins (SAE-CD), such as sulfobutylether cyclodextrin (SBECD).

These solubility-improved forms, also known as cyclodextrin derivatives, herein after referred to as “cyclodextrin/drug forms” can be simple physical mixtures. An example of such is found in U.S. Pat. No. 5,134,127, herein incorporated by reference. Alternatively, the drug and cyclodextrin may be complexed together. For example, the active drug and sulfoalkyl ether cyclodextrin (SAE-CD) may be preformed into a complex prior to the preparation of the final formulation. Alternatively, the drug can be formulated by using a film coating surrounding a solid core comprising a release rate modifier and a SAE-CD/drug mixture, as disclosed in U.S. Pat. No. 6,046,177, herein incorporated by reference. Alternatively, sustained-release formulations containing SAE-CD may consist of a core comprising a physical mixture of one or more SAE-CD derivatives, an optional release rate modifier, a therapeutic agent, a major portion of which is not complexed to the SAE-CD, and an optional release rate modifying coating surrounding the core. Other cyclodextrin/drug forms contemplated by the invention are found in U.S. Pat. Nos. 5,134,127, 5,874,418, and 5,376,645, all of which are incorporated by reference.

Another solubility-improved form of ziprasidone is a combination of ziprasidone and a solubilizing agent. Such solubilizing agents promote the aqueous solubility of ziprasidone. When ziprasidone is administered to an aqueous use environment in the presence of the solubilizing agent, the concentration of dissolved ziprasidone may exceed the equilibrium concentration of dissolved ziprasidone, at least temporarily. Examples of solubilizing agents include surfactants; pH control agents such as buffers, organic acids; glycerides; partial glycerides; glyceride derivatives; polyoxyethylene and polyoxypropylene ethers and their copolymers; sorbitan esters; polyoxyethylene sorbitan esters; alkyl sulfonates; and phospholipids. In this aspect, the drug and solubilizing agent are both preferably solid.

Exemplary surfactants include fatty acid and alkyl sulfonates; commercial surfactants such as benzalkonium chloride (HYAMINE® 1622, available from Lonza, Inc., Fairlawn, N.J.); dioctyl sodium sulfosuccinate (DOCUSATE SODIUM, available from Mallinckrodt Spec. Chem., St. Louis, Mo.); polyoxyethylene sorbitan fatty acid esters (TWEEN®, available from ICI Americas Inc., Wilmington, Del.; LIPOSORB® 0-20, available from Lipochem Inc., Patterson N.J.; CAPMUL® POE-0, available from Abitec Corp., Janesville, Wis.); and natural surfactants such as sodium taurocholic acid, 1-palmitoyl-2-oleoyl-sn-glycero-3-phosphocholine, lecithin, and other phospholipids and mono- and diglycerides.

One preferred class of solubilizing agents consists of organic acids. Exemplary organic acids include acetic, aconitic, adipic, ascorbic, aspartic, benzenesulfonic, benzoic, camphorsulfonic, cholic, citric, decanoic, erythorbic, 1,2-ethanedisulfonic, ethanesulfonic, formic, fumaric, gluconic, glucuronic, glutamic, glutaric, glyoxylic, heptanoic, hippuric, hydroxyethanesulfonic, lactic, lactobionic, levulinic, lysine, maleic, malic, malonic, mandelic, methanesulfonic, mucic, 1- and 2-naphthalenesulfonic, nicotinic, pamoic, pantothenic, phenylalanine, 3-phenylpropionic, phthalic, salicylic, saccharic, succinic, tannic, tartaric, p-toluenesulfonic, tryptophan, and uric.

Another class of solubilizing agents consists of lipophilic microphase-forming materials described in US published patent application 2003/0228358A1 published Dec. 11, 2003 herein incorporated by reference. Lipophilic microphase-forming material may comprise a surfactant and/or a lipophilic material. Thus, as used herein, the “lipophilic microphase-forming material” is intended to include blends of materials in addition to a single material. Examples of amphiphilic materials suitable for use as the lipophilic microphase-forming material include: sulfonated hydrocarbons and their salts, such as sodium 1,4-bis(2-ethylhexyl) sulfosuccinate, also known as docusate sodium (CROPOL) and sodium lauryl sulfate (SLS); poloxamers, also referred to as polyoxyethylene-polyoxypropylene block copolymers (PLURONICs, LUTROLs); polyoxyethylene alkyl ethers (CREMOPHOR A, BRIJ); polyoxyethylene sorbitan fatty acid esters (polysorbates, TWEEN); short-chain glyceryl mono-alkylates (HODAG, IMWITTOR, MYRJ); polyglycolized glycerides (GELUCIREs); mono- and di-alkylate esters of polyols, such as glycerol; nonionic surfactants such as polyoxyethylene 20 sorbitan monooleate, (polysorbate 80, sold under the trademark TWEEN 80, available commercially from ICI); polyoxyethylene 20 sorbitan monolaurate (Polysorbate 20, TWEEN 20); polyethylene (40 or 60) hydrogenated castor oil (available under the trademarks CREMOPHOR® RH40 and RH60 from BASF); polyoxyethylene (35) castor oil (CREMOPHOR® EL); polyethylene (60) hydrogenated castor oil (Nikkol HCO-60); alpha tocopheryl polyethylene glycol 1000 succinate (Vitamin E TPGS); glyceryl PEG 8 caprylate/caprate (available commercially under the registered trademark LABRASOL® from Gattefosse); PEG 32 glyceryl laurate (sold commercially under the registered trademark GELUCIRE 44/14 by Gattefosse), polyoxyethylene fatty acid esters (available commercially under the registered trademark MYRJ from ICI), polyoxyethylene fatty acid ethers (available commercially under the registered trademark BRIJ from ICI). Alkylate esters of polyols may be considered amphiphilic or hydrophobic depending on the number of alkylates per molecule and the number of carbons in the alkylate. When the polyol is glycerol, mono- and di-alkylates are often considered amphiphilic while trialkylates of glycerol are generally considered hydrophobic. However, some scientists classify even medium chain mono- and di-glycerides as hydrophobic. See for example Patel et al U.S. Pat. No. 6,294,192 (B1), which is incorporated herein in its entirety by reference. Regardless of the classification, compositions comprising mono- and di-glycerides are preferred compositions of this invention. Other suitable amphiphilic materials may be found in Patel, U.S. Pat. No. 6,294,192 and are listed as “hydrophobic non-ionic surfactants and hydrophilic ionic surfactants.”

It should be noted that some amphiphilic materials may not be water immiscible by themselves, but instead are at least somewhat water soluble. Such amphiphilic materials may nevertheless be used in mixtures to form the lipophilic microphase, particularly when used as mixtures with hydrophobic materials.

Examples of hydrophobic materials suitable for use as the lipophilic microphase-forming material include: medium-chain glyceryl mono-, di-, and tri-alkylates (CAPMUL MCM, MIGLYOL 810, MYVEROL 18-92, ARLACEL 186, fractionated coconut oil, light vegetable oils); sorbitan esters (ARLACEL 20, ARLACEL 40); long-chain fatty alcohols (stearyl alcohol, cetyl alcohol, cetostearyl alcohol); long-chain fatty-acids (stearic acid); and phospholipids (egg lecithin, soybean lecithin, vegetable lecithin, sodium taurocholic acid, and 1,2-diacyl-sn-glycero-3-phosphocholine, such as 1-palmitoyl-2-oleyl-sn-glycero-3-phosphocoline, 1,2-dipalmitoyl-sn-glycero-3-phosphocholine, 1,2-distearoyl-sn-glycero-3-phosphocholine, 1-palmitoyl-2-stearoyl-sn-glycero-3-phosphocholine, and other natural or synthetic phosphatidyl cholines); mono and diglycerides of capric and caprylic acid under the following registered trademarks: Capmul® MCM, MCM 8, and MCM 10, available commercially from Abitec, and Imwitor® 988, 742 or 308, available commercially from Condea Vista; polyoxyethylene 6 apricot kernel oil, available under the registered trademark Labrafil® M 1944 CS from Gattefosse; polyoxyethylene corn oil, available commercially as Labrafil® M 2125; propylene glycol monolaurate, available commercially as Lauroglycol from Gattefosse; propylene glycol dicaprylate/caprate available commercially as Captex® 200 from Abitec or Miglyol® 840 from Condea Vista, polyglyceryl oleate available commercially as Plurol oleique from Gattefosse, sorbitan esters of fatty acids (e.g., Span® 20, Crill® 1, Crill® 4, available commercially from ICI and Croda), and glyceryl monooleate (Maisine, Peceol); medium chain triglycerides (MCT, C6-C12) and long chain triglycerides (LCT, C14-C20) and mixtures of mono-, di-, and triglycerides, or lipophilic derivatives of fatty acids such as esters with alkyl alcohols; fractionated coconut oils, such as Miglyol® 812 which is a 56% caprylic (C8) and 36% capric (C10) triglyceride, Miglyol® 810 (68% C8 and 28% C10), Neobee® M5, Captex® 300, Captex® 355, and Crodamol® GTCC; (Miglyols are supplied by Condea Vista Inc. (Huls), Neobee® by Stepan Europe, Voreppe, France, Captex by Abitec Corp., and Crodamol by Croda Corp); vegetable oils such as soybean, safflower, corn, olive, cottonseed, arachis, sunflowerseed, palm, or rapeseed; fatty acid esters of alkyl alcohols such as ethyl oleate and glyceryl monooleate. Other hydrophobic materials suitable for use as the lipophilic microphase-forming material include those listed in Patel, U.S. Pat. No. 6,294,192 as “hydrophobic surfactants.” Exemplary classes of hydrophobic materials include: fatty alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; and mixtures thereof. Mixtures of relatively hydrophilic materials, such as those termed herein as “amphiphilic” or in Patel as “hydrophilic surfactants” and the above hydrophobic materials are particularly suitable. Specifically, the mixtures of hydrophobic surfactants and hydrophilic surfactants disclosed by Patel are suitable and for many compositions, preferred. However, unlike Patel, mixtures that include triglycerides as a hydrophobic component are also suitable.

In one embodiment, the lipophilic microphase-forming material is selected from the group consisting of polyglycolized glycerides (GELUCIREs); polyethylene (40 or 60) hydrogenated castor oil (available under the trademarks CREMOPHOR® RH40 and RH60 from BASF); polyoxyethylene (35) castor oil (CREMOPHOR® EL); polyethylene (60) hydrogenated castor oil (Nikkol HCO-60); alpha tocopheryl polyethylene glycol 1000 succinate (Vitamin E TPGS); glyceryl PEG 8 caprylate/caprate (available commercially under the registered trademark LABRASOL® from Gattefosse); PEG 32 glyceryl laurate (sold commercially under the registered trademark GELUCIRE 44/14 by Gattefosse); polyoxyethylene fatty acid esters (available commercially under the registered trademark MYRJ from ICI); polyoxyethylene fatty acid ethers (available commercially under the registered trademark BRIJ from ICI); polyoxyethylene-polyoxypropylene block copolymers (PLURONICs, LUTROLs); polyoxyethylene alkyl ethers (CREMOPHOR A, BRIJ); long-chain fatty alcohols (stearyl alcohol, cetyl alcohol, cetostearyl alcohol); long-chain fatty-acids (stearic acid); polyoxyethylene 6 apricot kernel oil, available under the registered trademark Labrafil® M 1944 CS from Gattefosse; polyoxyethylene corn oil, available commercially as Labrafil® M 2125; propylene glycol monolaurate, available commercially as Lauroglycol from Gattefosse; polyglyceryl oleate available commercially as Plurol oleique from Gattefosse; triglycerides, including medium chain triglycerides (MCT, C₆-C₁₂) and long chain triglycerides (LCT, C₁₄-C₂₀); fractionated coconut oils, such as Miglyol® 812 which is a 56% caprylic (C₈) and 36% capric (C₁₀) triglyceride, Miglyol® 810 (68% C₈ and 28% C₁₀), Neobee® M5, Captex® 300, Captex® 355, and Crodamol® GTCC; (Miglyols are supplied by Condea Vista Inc. [Huls], Neobee® by Stepan Europe, Voreppe, France, Captex by Abitec Corp., and Crodamol by Croda Corp); vegetable oils such as soybean, safflower, corn, olive, cottonseed, arachis, sunflowerseed, palm, or rapeseed; polyoxyethylene alkylethers; fatty acids; lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; transesterified vegetable oils; sterols; sterol derivatives; sugar esters; sugar ethers; sucroglycerides; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; and mixtures thereof.

Especially preferred lipophilic microphase-forming materials include mixtures of polyethoxylated castor oils and medium-chain glyceryl mono-, di-, and/or tri-alkylates, (such as mixtures of CREMOPHOR RH40 and CAPMUL MCM), mixtures of polyoxyethylene sorbitan fatty acid esters and medium-chain glyceryl mono-, di-, and/or tri-alkylates, (such as mixtures of TWEEN 80 and CAPMUL MCM), mixtures of polyethoxylated castor oils and medium-chain glyceryl mono-, di-, and/or tri-alkylates, (such as mixtures of CREMOPHOR RH40 and ARLACEL 20), mixtures of sodium taurocholic acid and palmitoyl-2-oleyl-sn-glycero-3-phosphocholine and other natural or synthetic phosphatidylcholines, and mixtures of polyglycolized glycerides and medium-chain glyceryl mono-, di-, and/or tri-alkylates, (such as mixtures of Gelucire 44/14 and CAPMUL MCM).

Yet another solubility-improved form of ziprasidone is ziprasidone in amorphous form. Preferably, at least a major portion of the ziprasidone is amorphous. By “amorphous” is meant simply that the ziprasidone is in a non-crystalline state. As used herein, the term “a major portion” of means that at least 60 wt % of the drug in the dosage form is in the amorphous form, rather than the crystalline form. Preferably, the ziprasidone is substantially amorphous. As used herein, “substantially amorphous” means that the amount of ziprasidone in crystalline form does not exceed about 25 wt %. More preferably, the ziprasidone is “almost completely amorphous,” meaning that the amount of ziprasidone in the crystalline form does not exceed about 10 wt %. Amounts of crystalline ziprasidone may be measured by Powder X-Ray Diffraction (PXRD), Scanning Electron Microscope (SEM) analysis, differential scanning calorimetry (DSC), or any other standard quantitative measurement.

The amorphous form of ziprasidone may be in any form in which ziprasidone is amorphous. Examples of amorphous forms of ziprasidone include solid amorphous dispersions of ziprasidone in a polymer, such as disclosed in commonly assigned US published patent application 2002/0009494A1 herein incorporated by reference. Alternatively, ziprasidone may be adsorbed in amorphous form on a solid substrate, such as disclosed in commonly assigned US published patent application 2003/0054037A1, herein incorporated by reference. As yet another alternative, amorphous ziprasidone may be stabilized using a matrix material, such as disclosed in commonly assigned US Patent application 2003/0104063A1, herein incorporated by reference.

Another solubility-improved form of ziprasidone is ziprasidone in a semi-ordered state, such as disclosed in commonly assigned U.S. Provisional Patent Application Ser. No. 60/403,087 filed Aug. 12, 2002, herein incorporated by reference.

Several methods, such as an in vitro dissolution test or a membrane permeation test may be used to determine if a form of ziprasidone is a solubility-improved form and the degree of solubility improvement. An in vitro dissolution test may be performed by adding the solubility-improved form of ziprasidone to a dissolution test media, such as model fasted duodenal (MFD) solution, phosphate buffered saline (PBS) solution, simulated intestinal buffer solution, or water and agitating to promote dissolution. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine. Appropriate simulated intestinal buffer solutions include (1) 50 mM NaH₂PO₄ and 2 wt % sodium lauryl sulfate, adjusted to pH 7.5, (2) 50 mM NaH₂PO₄ and 2 wt % sodium lauryl sulfate, adjusted to pH 6.5, and (3) 6 mM NaH₂PO₄, 150 mM NaCl, and 2 wt % sodium lauryl sulfate, adjusted to pH 6.5. Water is a preferred dissolution media for some fast precipitating salts. In one method for evaluating whether the form is a solubility-improved form, the solubility-improved form of ziprasidone when tested in an in vitro dissolution test meets at least one, and preferably both, of the following conditions. The first condition is that the solubility-improved form provides a higher maximum dissolved drug concentration (MDC) of ziprasidone in the in vitro dissolution test relative to a control composition consisting of the crystalline free base form of ziprasidone. That is, once the solubility-improved form is introduced into a use environment, the solubility-improved form provides a higher aqueous concentration of dissolved ziprasidone relative to the control composition. The control composition is the bulk crystalline form of ziprasidone free base alone. It is important to note that the solubility-improved form is dissolution tested independently of the dosage form so that the sustained release means do not interfere with evaluation of the degree of solubility improvement. Preferably, the solubility-improved form provides an MDC of ziprasidone in aqueous solution that is at least 1.25-fold that of the control composition, more preferably at least 2-fold, and most preferably at least 3-fold. For example, if the MDC provided by the test composition is 22 μg/ml, and the MDC provided by the control composition is 2 μg/ml, the solubility-improved form provides an MDC that is 11-fold that provided by the control composition.

The second condition is that the solubility-improved form provides a higher dissolution area under the concentration versus time curve (AUC) of dissolved ziprasidone in the in vitro dissolution test relative to a control composition consisting of an equivalent amount of crystalline ziprasidone free base alone. More specifically, in the in vitro use environment, the solubility-improved form provides an AUC for any 90-minute period from about 0 to about 270 minutes following introduction to the use environment that is at least 1.25-fold that of the control composition described above. Preferably, the AUC provided by the composition is at least 2-fold, more preferably at least 3-fold that of the control composition.

An in vitro test to evaluate enhanced ziprasidone concentration in aqueous solution can be conducted by (1) adding with agitation a sufficient quantity of control composition, that is, the crystalline ziprasidone free base alone, to the in vitro test medium, such as an MFD, PBS, or simulated intestinal buffer solution, to achieve equilibrium concentration of ziprasidone; (2) in a separate test, adding with agitation a sufficient quantity of test composition (e.g., the solubility-improved form) in the same test medium, such that if all ziprasidone dissolved, the theoretical concentration of ziprasidone would exceed the equilibrium concentration provided by crystalline ziprasidone free base by a factor of at least 2, and preferably by a factor of at least 10; and (3) comparing the measured MDC and/or aqueous AUC of the test composition in the test medium with the equilibrium concentration, and/or with the aqueous AUC of the control composition. In conducting such a dissolution test, the amount of test composition or control composition used is an amount such that if all of ziprasidone dissolved, the ziprasidone concentration would be at least 2-fold, preferably at least 10-fold, and most preferably at least 100-fold that of the equilibrium concentration.

The concentration of dissolved ziprasidone is typically measured as a function of time by sampling the test medium and plotting ziprasidone concentration in the test medium vs. time so that the MDC can be ascertained. The MDC is taken to be the maximum value of dissolved ziprasidone measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, (in less than about 30 minutes), the time interval used to calculate AUC is from time equals zero to time equals 90 minutes. However, if the AUC of a composition over any 90-minute time period described above meets the criterion of this invention, then the ziprasidone is considered to be in a solubility-improved form.

To avoid large drug particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred solubility-improved forms. It should be recognized that this definition of “dissolved drug” encompasses not only monomeric solvated drug molecules but also a wide range of species such as polymer/drug assemblies that have submicron dimensions such as drug aggregates, aggregates of mixtures of polymer and drug, micelles, polymeric micelles, colloidal particles or nanocrystals, polymer/drug complexes, and other such drug-containing species that are present in the filtrate or supernatant in the specified dissolution test.

In another method for evaluation of whether a drug form is a solubility-improved form, the dissolution rate of the solubility improved form is measured and compared to the dissolution rate of the free base form of ziprasidone having an average particle size of 10 μm. The dissolution rate may be tested in any appropriate dissolution media, such as PBS solution, MFD solution, simulated intestinal buffer solution, or distilled water. Distilled water is a preferred dissolution media for salt forms that rapidly precipitate. The dissolution rate of the solubility-improved form is greater than the dissolution rate of the free base form of ziprasidone having an average particle size of 10 μm. Preferably, the dissolution rate is 1.25-fold that of the free base form of ziprasidone, more preferably at least 2-fold that of the free base, and even more preferably at least 3-fold that of the free base.

Alternatively, an in vitro membrane-permeation test may be used to determine if ziprasidone is in a solubility-improved form. In this test, the solubility-improved form is placed in, dissolved in, suspended in, or otherwise delivered to the aqueous solution to form a feed solution. The aqueous solution can be any physiologically relevant solution, such as an MFD or PBS or simulated intestinal buffer solution, as described above. After forming the feed solution, the solution may be agitated to dissolve or disperse the solubility-improved form therein or may be added immediately to a feed solution reservoir. Alternatively, the feed solution may be prepared directly in a feed solution reservoir. Preferably, the feed solution is not filtered or centrifuged after administration of the solubility-improved form prior to performing the membrane-permeation test.

The feed solution is then placed in contact with the feed side of a microporous membrane, the feed side surface of the microporous membrane being hydrophilic. The portion of the pores of the membrane that are not hydrophilic are filled with an organic fluid, such as a mixture of decanol and decane, and the permeate side of the membrane is in fluid communication with a permeate solution comprising the organic fluid. Both the feed solution and the organic fluid remain in contact with the microporous membrane for the duration of the test. The length of the test may range from several minutes to several hours or even days.

The rate of transport of drug from the feed solution to the permeate solution is determined by measuring the concentration of drug in the organic fluid in the permeate solution as a function of time or by measuring the concentration of drug in the feed solution as a function of time, or both. This can be accomplished by methods well known in the art, including by use of ultraviolet/visible (UV/Vis) spectroscopic analysis, high-performance liquid chromatography (HPLC), gas chromatography (GC), nuclear magnetic resonance (NMR), infra red (IR) spectroscopic analysis, polarized light, density, and refractive index. The concentration of drug in the organic fluid can be determined by sampling the organic fluid at discrete time points and analyzing for drug concentration or by continuously analyzing the concentration of drug in the organic fluid. For continuous analysis, UV/Vis probes may be used, as can flow-through cells. In all cases, the concentration of drug in the organic fluid is determined by comparing the results against a set of standards, as well known in the art.

From these data, the maximum flux of drug across the membrane is calculated by multiplying the maximum slope of the concentration of drug in the permeate solution versus time plot by the permeate volume and dividing by the membrane area. This maximum slope is typically determined during the first 10 to 90 minutes of the test, where the concentration of drug in the permeate solution often increases at a nearly constant rate following a short time lag of a few minutes. At longer times, as more of the drug is removed from the feed solution, the slope of the concentration versus time plot decreases. Often, the slope approaches zero as the driving force for transport of drug across the membrane approaches zero; that is, the drug in the two phases approaches equilibrium. The maximum flux is determined either from the linear portion of the concentration versus time plot, or is estimated from a tangent to the concentration versus time plot at time where the slope is at its highest value if the curve is non-linear. Further details of this membrane-permeation test are presented in co-pending U.S. Patent Application Ser. No. 60/557,897, entitled “Method and Device for Evaluation of Pharmaceutical Compositions,” filed Mar. 30, 2004 (attorney Docket No. PC25968), incorporated herein by reference.

A typical in vitro membrane-permeation test to evaluate solubility-improved drug forms can be conducted by (1) administering a sufficient quantity of test composition (that is, the solubility-improved ziprasidone) to a feed solution, such that if all of the drug dissolved, the theoretical concentration of drug would exceed the equilibrium concentration of the drug by a factor of at least 2; (2) in a separate test, adding an equivalent amount of control composition (that is, crystalline ziprasidone free base) to an equivalent amount of test medium; and (3) determining whether the measured maximum flux of drug provided by the test composition is at least 1.25-fold that provided by the control composition. A composition is a solubility-improved form of ziprasidone if, when dosed to an aqueous use environment, it provides a maximum flux of drug in the above test that is at least about 1.25-fold the maximum flux provided by the control composition. Preferably, the maximum flux provided by the compositions are at least about 1.5-fold, more preferably at least about 2-fold, and even more preferably at least about 3-fold that provided by the control composition.

### Release Profile

The sustained release oral dosage forms release at least a portion of the ziprasidone from the dosage form after about 2 hours after administration to a use environment. In other words, the dosage forms do not release all of the ziprasidone immediately. By “immediate release” is meant that a dosage form releases greater than 90 wt % of all of the ziprasidone in the dosage form within the first two hours following administration. In one embodiment, the sustained release dosage form releases no greater than 90 wt % of the ziprasidone from the dosage form during the first 2 hours after administration to an in vitro use environment. In other embodiments, the dosage form releases no greater than 80 wt %, no greater than 70 wt %, or even no greater than about 60 wt % of the ziprasidone during the first 2 hours after administration to a use environment. The time to release at least 80 wt % of ziprasidone from the dosage form may be at least 4 hours, at least 6 hours, at least 8 hours, at least 10 hours, or even at least 12 hours. By “release” is meant the amount of ziprasidone that exits or is released by the dosage form, rather than the amount of ziprasidone that is dissolved in the use environment. Thus, for example, the dosage form may release ziprasidone that is crystalline (not dissolved) into the use environment, which then dissolves subsequent to release.

An in vitro test may be used to determine whether a dosage form releases at least a portion of the ziprasidone from the dosage form after about 2 hours after administration to a use environment. In vitro tests are well known in the art. The in vitro tests are designed to approximate the behavior of the dosage form in vivo. One such test is a “residual test,” which is performed as follows. A plurality of dosage forms are each placed into separate stirred USP type 2 dissolution flasks containing 900 mL of 0.05 M sodium dihydrogen phosphate, pH 6.5, with 2 wt % sodium lauryl sulfate, at 37° C. simulating an intestinal environment. The dosage form is placed in the dissolution medium, and the medium is stirred using paddles that rotate at a rate of 75 rpm. When the dosage form is in the form of a tablet, capsule or other solid dosage form, the dosage form may be placed in a wire support to keep the dosage form off of the bottom of the flask, so that all of its surfaces are exposed to the dissolution media. After a given time interval, a dosage form is removed from a flask, material adhering to the surface is wiped away from the surface of the dosage form, and the dosage form cut in half and placed in 100 mL of a recovery solution as follows. For the first two hours, the dosage form is stirred in 25 mL acetone or other solvent suitable to dissolve any coating on the dosage form. Next, 75 mL of methanol is added and stirring continued overnight at ambient temperature to dissolve the drug remaining in the dosage form. Approximately 2 mL of the recovery solution is removed and centrifuged, and 250 μL of supernatant added to an HPLC vial and diluted with 750 μL methanol. Residual drug is then analyzed by HPLC. HPLC analysis is performed using a Zorbax RxC8 Reliance column. The mobile phase consists of 55% 50 mM potassium dihydrogen phosphate, pH 6.5 and 45% acetonitrile. UV absorbance is measured at 315 nm. The amount of drug remaining in the dosage form is subtracted from the total drug initially present in the dosage form to obtain the amount released at each time interval.

The dosage forms of the present invention may also be evaluated using a so-called “direct” test, where the dosage form is placed into a stirred USP type 2 dissolution flask containing 900 mL of 0.05 M sodium dihydrogen phosphate, pH 6.5, with 2 wt % sodium lauryl sulfate, at 37° C. simulating an intestinal environment as previously described. The dosage form is placed in a wire support in the dissolution medium, and the medium is stirred using paddles that rotate at a rate of 75 rpm. Samples of the dissolution medium are taken at periodic intervals, for example, by using a VanKel VK8000 autosampling dissoette with automatic receptor solution replacement. The concentration of released drug in the dissolution medium is then determined by HPLC, as described above. (In some cases the released ziprasidone may not be sufficiently solubilized to be completely dissolved. In such cases, the released suspended ziprasidone contained in the sample is dissolved and then assayed). The mass of released drug in the dissolution medium is then calculated from the concentration of drug in the medium and the volume of the medium, and expressed as a percentage of the mass of drug originally present in the dosage form.

In some embodiments, the sustained release dosage form may provide certain blood levels of ziprasidone following administration.

In one aspect, the sustained release dosage form provides a steady state minimum blood ziprasidone concentration. The sustained release dosage form provides a minimum steady state blood ziprasidone concentration in the blood (Cₘᵢₙ) of at least 20 ng/ml after administration in the fed state either once or twice a day. By “steady state” is meant the state achieved after administration of the dosage form over a sufficient period of time (e.g., from three days to a week) so that the maximum and minimum ziprasidone concentrations in the blood have plateaued (that is, reached a relatively constant value). (Of course, reference to administration of a dosage form means dosage forms having the same composition are administered once or twice a day to achieve steady state, and not that a single dosage form is repeatedly administered). Preferably, the sustained release dosage form provides a steady state minimum concentration of ziprasidone in the blood of at least 30 ng/ml, and more preferably at least 50 ng/ml.

The sustained release dosage forms also limit the maximum steady state blood ziprasidone concentration (Cₘₐₓ). The sustained release dosage form provides a maximum steady state blood ziprasidone concentration in the blood of less than 330 ng/ml after administration in the fed state when administered either once or twice a day. Preferably, the sustained release dosage form provides a steady state maximum concentration of ziprasidone in the blood of less than 265 ng/ml, and more preferably less than 200 ng/ml.

In a preferred embodiment, the dosage form limits the steady state ratio of Cₘₐₓ to Cₘᵢₙ. In one embodiment, when the sustained release dosage form is dosed twice per day, the sustained release dosage form provides a steady state ratio of the maximum concentration of ziprasidone in the blood (Cₘₐₓ) to the minimum concentration of ziprasidone in the blood (Cₘᵢₙ) that is less than about 2.6. By keeping the ratio of Cₘₐₓ to Cₘᵢₙ low, the sustained release dosage form may provide a more uniform patient response, and may reduce or mitigate side effects relative to an immediate release dosage form containing the same amount of ziprasidone. In a more preferred embodiment, the steady state ratio of Cₘₐₓ to Cₘᵢₙ is less than about 2.4, and even more preferably less than about 2.2, when dosed twice per day. In another embodiment, when dosed only once per day, the sustained release dosage form provides a steady state ratio of the maximum concentration of ziprasidone in the blood (Cₘₐₓ) to the minimum concentration of ziprasidone in the blood (Cₘᵢₙ) that is less than about 12. In a more preferred embodiment, the steady state ratio of Cₘₐₓ to Cₘᵢₙ is less than about 10, and even more preferably is less than about 8 when dosed only once per day.

In another aspect, the sustained release dosage form provides a steady state area under the concentration of ziprasidone in the blood versus time curve after administration in the fed state. For those dosage forms that are administered twice daily, the steady state AUC_{0-T} (where T is the dosing interval) is preferably at least 240 ng-hr/ml, more preferably at least 420 ng-hr/ml, and even more preferably at least 600 ng-hr/ml. For those dosage forms administered once per day, the sustained release dosage form preferably provides a steady state AUC_{0-T} after administration in the fed state that is at least 480 ng-hr/ml, more preferably at least 840 ng-hr/ml, and even more preferably at least 1200 ng-hr/ml.

In some embodiments, the sustained release dosage forms may provide improvement relative to the IR oral capsule.

In one aspect, the sustained release dosage form reduces the steady state ratio of Cₘₐₓ to Cₘᵢₙ relative to that provided by a control IR oral capsule when administered at the same dosing interval. By “control IR oral capsule” is meant the commercially available GEODON™ capsules for oral administration manufactured by Pfizer, Inc. containing the same amount of active ziprasidone. GEODON™ capsules contain ziprasidone hydrochloride monohydrate, lactose, pregelatinized starch, and magnesium stearate. (If the commercial GEODON capsule is unavailable, the control IR oral capsule means a capsule that releases greater than 95 wt % of ziprasidone within two hours following administration to the dissolution test media described in the dissolution test exemplified in the In Vitro Release Tests of the Examples as reported in Table 6). More preferably, the steady state ratio of Cₘₐₓ to Cₘᵢₙ provided by the sustained release dosage form is less than 90% that of the control immediate release oral capsule, and even more preferably is less than 80% that of the control immediate release oral capsule. Lowering the steady state ratio of Cₘₐₓ to Cₘᵢₙ has the advantage of allowing the sustained release dosage forms to either contain greater amounts of ziprasidone (relative to the IR oral capsule) and result in higher doses without increasing the maximum ziprasidone blood concentrations, or contain the same amount of ziprasidone (relative to the IR oral capsule) but lower the maximum ziprasidone blood concentration.

It is also desired that while the dosage forms reduce the ratio of Cₘₐₓ to Cₘᵢₙ, the dosage forms do not substantially decrease the relative bioavailability of ziprasidone. Thus, in yet another aspect, the sustained release dosage forms of the present invention preferably provide a relative bioavailability when administered to a human patient in the fed state of at least 50% relative to a control IR oral capsule containing the same amount of ziprasidone. In a more preferred embodiment, the sustained release dosage form may provide a relative bioavailability of at least 60% relative to the immediate release capsule. In an even more preferred embodiment, the sustained release dosage form provides a relative bioavailability is at least 70% relative to the immediate release capsule.

The Cₘₐₓ, Cₘᵢₙ, Cₘₐₓ/Cₘᵢₙ ratio, and relative bioavailability of ziprasidone provided by the sustained release dosage forms can be tested in vivo in humans using conventional methods for making such a determination. An in vivo test, such as a crossover study, may be used to determine the relative bioavailability of the sustained release dosage form compared with the control IR oral capsule containing the same amount of active ziprasidone. In an in vivo crossover study a test sustained release dosage form is dosed to half a group of test subjects and, after an appropriate washout period (e.g., one week) the same subjects are dosed with the control IR oral capsule that consists of an equivalent quantity of ziprasidone. The other half of the group is dosed with the IR oral capsule first, followed by the test sustained release dosage form. The relative bioavailability is measured as the concentration of ziprasidone in the blood (serum or plasma) versus time area under the curve (AUC) determined for the test group divided by the AUC in the blood provided by the control IR oral capsule. Preferably, this test/control ratio is determined for each subject, and then the ratios are averaged over all subjects in the study. In vivo determinations of AUC can be made by plotting the serum or plasma concentration of drug along the ordinate (y-axis) against time along the abscissa x-axis). Methods for determining the AUCs and the relative bioavailability of a dosage form are well known in the art. (The calculation of an AUC is a well-known procedure in the pharmaceutical arts and is described, for example, in Welling, “Pharmacokinetics Processes and Mathematics,” ACS Monograph 185 (1986)).

Ziprasidone blood concentrations and relative bioavailability are measured after administration of the sustained release dosage form and the immediate release control oral dosage form in the fed state. By “fed state” is meant after a meal as is known by those skilled in the art. For example, administration in the fed state may be administration after a “standard” breakfast consisting of 2 eggs fried in butter, 2 strips of bacon, 2 ounces of hash brown potatoes, 2 slices of white toast with 2 pats of butter, and 240 mL of whole milk. The entire meal is to be consumed within 20 minutes prior to receiving the dosage form.

### Precipitation Inhibitors

For those embodiments which release ziprasidone over a long period of time, particularly those that allow once a day administration of the sustained release dosage form, the sustained release dosage form releases ziprasidone in a form and manner that facilitates absorption from the lumen of the intestines. In these embodiments, the dosage form contains ziprasidone in a solubility-improved form, and a precipitation inhibitor to improve the concentration of dissolved ziprasidone in the use environment.

By a “precipitation inhibitor” is meant any material known in the art that is capable of slowing the rate at which ziprasidone crystallizes or precipitates from an aqueous solution that is supersaturated with ziprasidone. Precipitation inhibitors suitable for use in the sustained release dosage forms of the present invention should be inert, in the sense that they do not chemically react with ziprasidone in an adverse manner, be pharmaceutically acceptable, and have at least some solubility in aqueous solution at physiologically relevant pHs (e.g. 1-8). The precipitation inhibitor can be neutral or ionizable, and should have an aqueous-solubility of at least 0.1 mg/mL over at least a portion of the pH range of 1-8.

Precipitation inhibitors may be polymers or non-polymeric. Precipitation-inhibiting polymers suitable for use with the present invention may be cellulosic or non-cellulosic. The polymers may be neutral or ionizable in aqueous solution. Of these, ionizable and cellulosic polymers are preferred, with ionizable cellulosic polymers being more preferred.

A preferred class of polymers comprises polymers that are “amphiphilic” in nature, meaning that the polymer has hydrophobic and hydrophilic portions. The hydrophobic portion may comprise groups such as aliphatic or aromatic hydrocarbon groups. The hydrophilic portion may comprise either ionizable or non-ionizable groups that are capable of hydrogen bonding such as hydroxyls, carboxylic acids, esters, amines or amides.

One class of polymers suitable for use with the present invention comprises neutral non-cellulosic polymers. Exemplary polymers include: vinyl polymers and copolymers having substituents of hydroxyl, alkylacyloxy, or cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyoxyethylene-polyoxypropylene copolymers, also known as poloxamers; and polyethylene polyvinyl alcohol copolymers.

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Degussa, of Maiden, Mass.; amine-functionalized polyacrylates and polymethacrylates; proteins; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers, and polyoxyethylene-polyoxypropylene copolymers. Exemplary commercial grades of such copolymers include the EUDRAGITS, which are copolymers of methacrylates and acrylates, and the PLURONICS supplied by BASF, which are polyoxyethylene-polyoxypropylene copolymers.

A preferred class of polymers comprises ionizable and neutral cellulosic polymers with at least one ester- and/or ether-linked substituent in which the polymer has a degree of substitution of at least 0.1 for each substituent.

It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.1 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulosic polymer has a degree of substitution of at least one relatively hydrophobic substituent of at least 0.1. Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

A preferred set of neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A preferred class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic group may itself be ionizable, such as phthalate or trimellitate substituents.

Exemplary cellulosic polymers that are at least partially ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary cellulosic polymers that meet the definition of amphiphilic, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A particularly desirable subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxypropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another particularly desirable subset of cellulosic ionizable polymers are those that possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate, and carboxymethyl ethyl cellulose.

While, as listed above, a wide range of polymers may be used, the inventors have found that relatively hydrophobic polymers have shown the best performance as demonstrated by high MDC and AUC values. In particular, cellulosic polymers that are aqueous insoluble in their nonionized state but are aqueous soluble in their ionized state perform particularly well. A particular subclass of such polymers are the so-called “enteric” polymers, which include, for example, hydroxypropyl methyl cellulose acetate succinate (HPMCAS), hydroxypropyl methyl cellulose phthalate (HPMCP), cellulose acetate phthalate (CAP), cellulose acetate trimellitate (CAT), and carboxymethyl ethyl cellulose (CMEC). In addition, non-enteric grades of such polymers, as well as closely related cellulosic polymers, are expected to perform well due to the similarities in physical properties.

Thus, especially preferred polymers are hydroxypropyl methyl cellulose acetate succinate (HPMCAS), hydroxypropyl methyl cellulose phthalate (HPMCP), cellulose acetate phthalate (CAP), cellulose acetate trimellitate (CAT), methyl cellulose acetate phthalate, hydroxypropyl methyl cellulose acetate phthalate, cellulose acetate terephthalate, cellulose acetate isophthalate, and carboxymethyl ethyl cellulose. The most preferred ionizable cellulosic polymers are hydroxypropyl methyl cellulose acetate succinate, hydroxypropyl methyl cellulose phthalate, cellulose acetate phthalate, cellulose acetate trimellitate, and carboxymethyl ethyl cellulose.

While specific polymers have been discussed as being suitable for use in the compositions of the present invention, blends of such polymers may also be suitable. Thus the term “polymer” is intended to include blends of polymers in addition to a single species of polymer. In particular, it has been found that ionizable cellulosic polymers such as HPMCAS function best over particular pH ranges. For example, HPMCAS aqueous properties are a function of the degree of substitution of each of the substituents: hydroxypropoxy, methoxy, acetate, and succinate, as well as the pH of the use environment. For example, HPMCAS is manufactured by Shin-Etsu, and sold under the trade name AQOAT as three different grades that differ in their levels of substituents and therefore their properties as a function of pH. Thus, it has been found in in vitro tests, that the H grade of HPMCAS is preferred for inhibition of crystallization in a pH 6.5 use environment. The H grade of HPMCAS has 22-26 wt % methoxy, 6 10 wt % hydroxypropoxy, 10-14 wt % acetate, and 4-8 wt % succinate groups. At lower pH values, say 5 to 6, the M grade of HPMCAS is preferred. The M grade of HPMCAS has 21-25 wt % methoxy, 5-9 wt % hydroxypropoxy, 7-11 wt % acetate, and 10-14 wt % succinate groups. It has also been found that in a use environment where the pH may be variable, such as in the GI tract of a mammal, a mixture of two or more grades may be preferred. Specifically, the inventors have found that delivering a solubility improved form of ziprasidone, such as the chloride salt in micronized form, along with a crystallization inhibitor comprising a mixture of HPMCAS grades, such as a 1 to 1 mixture of the H grade and M grade of HPMCAS, to the GI tract of a mammal, yields excellent absorption of ziprasidone.

Another preferred class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer, that has a pKa of less than about 10. Exemplary classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.” Neutralized acidic polymers are described in more detail in commonly assigned copending U.S. patent application Ser. No. 10/175,566 entitled “Pharmaceutical Compositions of Drugs and Neutralized Acidic Polymers” filed Jun. 17, 2002, the relevant disclosure of which is incorporated by reference.

In addition, the preferred polymers listed above, that is amphiphilic cellulosic polymers, tend to have greater precipitation-inhibiting properties relative to the other polymers of the present invention. Generally those precipitation-inhibiting polymers that have ionizable substituents tend to perform best. In vitro tests of compositions with such polymers tend to have higher MDC and AUC values than compositions with other polymers of the invention.

Several methods, such as an in vitro dissolution test or a membrane permeation test may be used to evaluate precipitation inhibitors and the degree of concentration enhancement provided by the precipitation inhibitors. An in vitro dissolution test may be performed by adding the solubility-improved form of ziprasidone together with the precipitation inhibitor to MFD or PBS or simulated intestinal buffer solution and agitating to promote dissolution. To evaluate the utility of precipitation inhibitors in use environments at other pH values, it may be desirable to use other similar dissolution media that have pH values adjusted to other values. For example, an acid such as HCl or H₃PO₄ may be added to PBS or MFD to adjust the pH of the solution to 6.0 or 5.0 and then used in the following dissolution tests. A solubility-improved form of ziprasidone together with the precipitation inhibitor, when tested in an in vitro dissolution test meets at least one, and preferably both, of the following conditions. The first condition is that the solubility-improved form and precipitation inhibitor provide a higher maximum dissolved drug concentration (MDC) of ziprasidone in the in vitro dissolution test relative to a control composition. The control composition consists of the solubility-improved form of ziprasidone alone (without the precipitation inhibitor). That is, once the solubility-improved form and the precipitation inhibitor are introduced into a use environment, the solubility-improved form and precipitation inhibitor provide a higher aqueous concentration of dissolved ziprasidone relative to the control composition. It is important to note that the solubility-improved form and precipitation inhibitor are dissolution tested independently of the dosage form so that the sustained release means do not interfere with evaluation of the degree of solubility improvement. Preferably, the solubility-improved form and precipitation inhibitor provide an MDC of ziprasidone in aqueous solution that is at least 1.25-fold that of the control composition, more preferably at least 2-fold, and most preferably at least 3-fold. For example, if the MDC provided by the test composition is 5 μg/ml, and the MDC provided by the control composition is 1 μg/ml, the test composition provides an MDC that is 5 fold that provided by the control composition.

The second condition is that the solubility-improved form and precipitation inhibitor provide a higher dissolution area under the concentration versus time curve (AUC) of dissolved ziprasidone in the in vitro dissolution test relative to a control composition. More specifically, in the use environment, the solubility-improved form and precipitation inhibitor provide an AUC for any 90-minute period of from about 0 to about 270 minutes following introduction to the use environment that is at least 1.25-fold that of the control composition. Preferably, the AUC provided by the composition is at least 2-fold, more preferably at least 3-fold that of the control composition.

Alternatively, an in vitro membrane-permeation test may be used to evaluate the precipitation inhibitor. In this test, described above, the solubility-improved form and precipitation inhibitor are placed in, dissolved in, suspended in, or otherwise delivered to the aqueous solution to form a feed solution. A typical in vitro membrane-permeation test to evaluate precipitation inhibitors can be conducted by (1) administering a sufficient quantity of test composition (that is, the solubility-improved ziprasidone and precipitation inhibitor) to a feed solution, such that if all of the drug dissolved, the theoretical concentration of drug would exceed the equilibrium concentration of the drug by a factor of at least 3; (2) in a separate test, adding an equivalent amount of control composition to an equivalent amount of test medium; and (3) determining whether the measured maximum flux of drug provided by the test composition is at least 1.25-fold that provided by the control composition. The solubility-improved form and precipitation inhibitor, when dosed to an aqueous use environment, provide a maximum flux of drug in the above test that is at least about 1.25-fold the maximum flux provided by the control composition. Preferably, the maximum flux provided by the test composition is at least about 1.5-fold, more preferably at least about 2-fold, and even more preferably at least about 3-fold that provided by the control composition.

The sustained-release dosage forms of this embodiment comprise a combination of a solubility-improved form of ziprasidone and a precipitation-inhibiting polymer. “Combination” as used herein means that the solubility-improved form and precipitation-inhibiting polymer may be in physical contact with each other or in close proximity but without the necessity of being physically mixed. For example, the combination may be in the form of a multi-layer tablet, as known in the art, wherein one or more layers comprises the solubility-improved form and one or more different layers comprises the precipitation-inhibiting polymer. Yet another example may constitute a coated tablet wherein either the solubility-improved form of the drug or the precipitation-inhibiting polymer or both may be present in the tablet core and the coating may comprise the solubility-improved form or the precipitation-inhibiting polymer or both. Alternatively, the combination can be in the form of a simple dry physical mixture wherein both the solubility-improved form and precipitation-inhibiting polymer are mixed in particulate form and wherein the particles of each, regardless of size, retain the same individual physical properties that they exhibit in bulk. Any conventional method used to mix the polymer and drug together such as physical mixing and dry or wet granulation, may be used.

The combination of solubility-improved form and precipitation inhibitor may be prepared by dry- or wet-mixing the drug or drug mixture with the precipitation inhibitor to form the composition. Mixing processes include physical processing as well as wet-granulation and coating processes.

For example, mixing methods include convective mixing, shear mixing, or diffusive mixing. Convective mixing involves moving a relatively large mass of material from one part of a powder bed to another, by means of blades or paddles, revolving screw, or an inversion of the powder bed. Shear mixing occurs when slip planes are formed in the material to be mixed. Diffusive mixing involves an exchange of position by single particles. These mixing processes can be performed using equipment in batch or continuous mode. Tumbling mixers (e.g., twin-shell) are commonly used equipment for batch processing. Continuous mixing can be used to improve composition uniformity.

Milling may also be employed to prepare the compositions of the present invention. Milling is the mechanical process of reducing the particle size of solids (comminution). Because in some cases milling may alter crystalline structure and cause chemical changes for some materials, milling conditions are generally chosen which do not alter the physical form of the drug. The most common types of milling equipment are the rotary cutter, the hammer, the roller and fluid energy mills. Equipment choice depends on the characteristics of the ingredients in the drug form (e.g., soft, abrasive, or friable). Wet- or dry-milling techniques can be chosen for several of these processes, also depending on the characteristics of the ingredients (e.g. drug stability in solvent). The milling process may serve simultaneously as a mixing process if the feed materials are heterogeneous. Conventional mixing and milling processes suitable for use in the present invention are discussed more fully in Lachman, et al., The Theory and Practice of Industrial Pharmacy (3rd Ed. 1986). The components of the compositions of this invention may also be combined by dry- or wet-granulating processes.

In addition to the physical mixtures described above, the compositions of the present invention may constitute any device or collection of devices that accomplishes the objective of delivering to the use environment both the drug and the precipitation inhibitor. Thus, in the case of oral administration to a mammal, the dosage form may constitute a layered tablet wherein one or more layers comprise the drug and one or more other layers comprise the polymer. Alternatively, the dosage form may be a coated tablet wherein the tablet core comprises the drug and the coating comprises the polymer. In addition, the drug and the polymer may even be present in different dosage forms such as tablets or beads and may be administered simultaneously or separately as long as both the drug and polymer are administered in such a way that the drug and polymer can come into contact in the use environment. When the drug and the polymer are administered separately it is generally preferable to deliver the polymer prior to the drug.

In one preferred embodiment, the combination comprises particles of the solubility-improved form of ziprasidone coated with a precipitation-inhibiting polymer. The particles may be either ziprasidone crystals, or particles of some other solubility-improved form such as amorphous drug or a cyclodextrin complex. This embodiment finds particularly utility when it is desired to provide absorption of ziprasidone in the intestines, particularly the colon. Without wishing to be bound by theory, when the polymer and ziprasidone are released into the intestinal use environment, the polymer may begin to dissolve and gel prior to dissolution of the drug. Thus, as the drug dissolves into the intestinal use environment, the dissolved drug immediately encounters dissolved polymer surrounding the dissolved drug. This has the advantage of preventing nucleation of the drug, thus reducing the rate of precipitation of the drug.

The polymer may be coated around the ziprasidone crystals using any conventional method. A preferred method is a spray drying process. The term spray-drying is used conventionally and broadly refers to processes involving breaking up liquid mixtures or suspensions into small droplets (atomization) and rapidly removing solvent from the droplets in a container where there is a strong driving force for evaporation of solvent.

To coat the ziprasidone crystals by spray drying, first a suspension of ziprasidone crystals and dissolved polymer is formed in a solvent. The relative amounts of drug suspended in the solvent and polymer dissolved in the solvent are chosen to yield the desired drug to polymer ratio in the resulting particles. For example, if a particle having a drug to polymer ratio of 0.33 (25 wt % drug) is desired, then the spray solution comprises 1 part crystalline drug particles and 3 parts polymer dissolved in the solvent. The total solids content of the spray solution is preferably sufficiently high so that the spray solution results in efficient production of the particles. The total solids content refers to the amount of solid drug, dissolved polymer and other excipients dissolved in the solvent. For example, to form a spray solution having a 5 wt % dissolved solids content and which results in a particle having a 25 wt % drug loading, the spray solution would comprise 1.25 wt % drug, 3.75 wt % polymer and 95 wt % solvent. To achieve good yield, the spray solution preferably has a solids content of at least 3 wt %, more preferably at least 5 wt %, and even more preferably at least 10 wt %. However, the dissolved solids content should not be too high, or else the spray solution may be too viscous to atomize efficiently into small droplets.

Often it is desirable for the particle size of the ziprasidone to be relatively small. This promotes satisfactory coating of the ziprasidone particles by the polymer. Thus, it is generally preferred for the ziprasidone particles to have a volume average diameter of less than about 10 μm and preferably less than about 5 μm.

The solvent is chosen based on the following characteristics: (1) the drug is insoluble or only slightly soluble in the solvent; (2) the polymer is soluble in the solvent; and (3) the solvent is relatively volatile. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, THF, cyclic ethers, and 1,1,1-trichloroethane. A preferred solvent is acetone. Mixtures of solvents may also be used, as can mixtures with water as long as the polymer is sufficiently soluble to make the spray-drying process practicable. In some cases it may be desired to add a small amount of water to aid solubility of the polymer in the spray solution.

Spray drying to form polymer coatings around drug particles is well known and is described in, for example, U.S. Pat. No. 4,767,789, U.S. Pat. No. 5,013,537, and U.S. published patent application 2002/0064108A1, herein incorporated by reference.

Alternatively, the polymer may be coated around the drug crystals using a rotary disk atomizer, as described in U.S. Pat. No. 4,675,140, herein incorporated by reference.

Alternatively, the precipitation-inhibiting polymer may be sprayed onto the drug particles in a high shear mixer or a fluid bed.

The amount of precipitation inhibitor may vary widely. In general, the amount of precipitation inhibitor should be sufficient to provide concentration-enhancement of the drug relative to a control composition consisting of the drug alone as described above. The weight ratio of solubility-improved form to precipitation inhibitor may range from 100 to 0.01. Where the precipitation inhibitor is a polymer, good results are generally achieved where the polymer to drug weight ratio is at least 0.33 (at least 25 wt % polymer), more preferably at least 0.66 (at least 40 wt % polymer), and even more preferably at least 1 (at least 50 wt % polymer). However, since it is desired to limit the size of the dosage form, the amount of precipitation inhibitor may be less than the amount that provides the greatest degree of concentration enhancement.

### Sustained-Release Means

The oral dosage forms of the present invention provide sustained-release of ziprasidone. The means for providing sustained release of ziprasidone can be any dosage form or collection of dosage forms known in the pharmaceutical arts that allow delivery of a drug in a sustained manner. Exemplary dosage forms include erodible and non-erodible matrix sustained-release dosage forms, osmotic sustained-release dosage forms, multiparticulates, and enteric coated cores.

### Matrix Sustained Release Dosage Forms

In one embodiment, ziprasidone is incorporated into an erodible or non-erodible polymeric matrix sustained release dosage form. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous use environment, the erodible polymeric matrix imbibes water and forms an aqueous-swollen gel or “matrix” that entraps the ziprasidone. The aqueous-swollen matrix gradually erodes, swells, disintegrates, disperses or dissolves in the environment of use, thereby controlling the release of ziprasidone to the environment of use. Examples of such dosage forms are well known in the art. See, for example, Remington: The Science and Practice of Pharmacy, 20^{th} Edition, 2000. Examples of such dosage forms are also disclosed in commonly assigned pending U.S. patent application Ser. No. 09/495,059 filed Jan. 31, 2000 which claimed the benefit of priority of provisional patent application Ser. No. 60/119,400 filed Feb. 10, 1999, the relevant disclosure of which is herein incorporated by reference. Other examples are disclosed in U.S. Pat. No. 4,839,177 and U.S. Pat. No. 5,484,608, herein incorporated by reference.

The erodible polymeric matrix into which ziprasidone is incorporated may generally be described as a set of excipients that are mixed with ziprasidone that, when contacted with the aqueous environment of use imbibes water and forms a water-swollen gel or “matrix” that entraps the drug. Drug release may occur by a variety of mechanisms: the matrix may disintegrate or dissolve from around particles or granules of the drug; or the drug may dissolve in the imbibed aqueous solution and diffuse from the tablet, beads or granules of the dosage form. A key ingredient of this water-swollen matrix is the water-swellable, erodible, or soluble polymer, which may generally be described as an osmopolymer, hydrogel or water-swellable polymer. Such polymers may be linear, branched, or crosslinked. They may be homopolymers or copolymers. Although they may be synthetic polymers derived from vinyl, acrylate, methacrylate, urethane, ester and oxide monomers, they are most preferably derivatives of naturally occurring polymers such as polysaccharides or proteins. Exemplary materials include hydrophilic vinyl and acrylic polymers, polysaccharides such as calcium alginate, polyethylene oxide (PEO), polyethylene glycol (PEG), polypropylene glycol (PPG). Exemplary naturally occurring polymers include naturally occurring polysaccharides such as chitin, chitosan, dextran and pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; phosphatides such as lecithin; alginates such as ammonium alginate, sodium, potassium or calcium alginate, propylene glycol alginate; gelatin; collagen; and cellulosics. By “cellulosics” is meant a cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

A preferred class of cellulosics for the erodible matrix comprises aqueous-soluble and aqueous-erodible cellulosics such as ethyl cellulose (EC), methylethyl cellulose (MEC), carboxymethyl cellulose (CMC), carboxymethyl ethylcellulose (CMEC), hydroxyethyl cellulose (HEC), hydroxypropyl cellulose (HPC), cellulose acetate (CA), cellulose propionate (CPr), cellulose butyrate (CB), cellulose acetate butyrate (CAB), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC). A particularly preferred class of such cellulosics comprises various grades of low viscosity (MW less than or equal to 50,000 daltons) and high viscosity (MW greater than 50,000 daltons) HPMC. Commercially available low viscosity HPMC polymers include the Dow METHOCEL series E5, E15LV, E50LV and K100LY, while high viscosity HPMC polymers include E4MCR, E10MCR, K4M, K15M and K100M; especially preferred in this group are the METHOCEL (Trademark) K series. Other commercially available types of HPMC include the Shin Etsu METOLOSE 90SH series.

Although the primary role of the erodible matrix material is to control the rate of release of ziprasidone to the environment of use, the inventors have found that the choice of matrix material can have a large effect on the maximum drug concentration attained by the dosage form as well as the maintenance of a high drug concentration. In one embodiment, the matrix material is a precipitation-inhibiting polymer, as defined herein.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl) methacrylate chloride.

The erodible matrix polymer may also contain a wide variety of additives and excipients known in the pharmaceutical arts, including osmopolymers, osmagens, solubility-enhancing or -retarding agents and excipients that promote stability or processing of the dosage form.

Alternatively, the sustained-release means may be a non-erodible matrix dosage form. In such dosage forms, ziprasidone in a solubility-improved form is distributed in an inert matrix. The drug is released by diffusion through the inert matrix. Examples of materials suitable for the inert matrix include insoluble plastics, such as copolymers of ethylene and vinyl acetate, methyl acrylate-methyl methacrylate copolymers, polyvinyl chloride, and polyethylene; hydrophilic polymers, such as ethyl cellulose, cellulose acetate, and crosslinked polyvinylpyrrolidone (also known as crospovidone); and fatty compounds, such as carnauba wax, microcrystalline wax, and triglycerides. Such dosage forms are described further in Remington: The Science and Practice of Pharmacy, 20^{th} edition (2000).

Matrix sustained release dosage forms may be prepared by blending ziprasidone and other excipients together, and then forming the blend into a tablet, caplet, pill, or other dosage form formed by compressive forces. Such compressed dosage forms may be formed using any of a wide variety of presses used in the fabrication of pharmaceutical dosage forms. Examples include single-punch presses, rotary tablet presses, and multilayer rotary tablet presses, all well known in the art. See for example, Remington: The Science and Practice of Pharmacy, 20^{th} Edition, 2000. The compressed dosage form may be of any shape, including round, oval, oblong, cylindrical, or triangular. The upper and lower surfaces of the compressed dosage form may be flat, round, concave, or convex.

When formed by compression, the dosage form preferably has a “strength” of at least 5 Kiloponds (kp)/cm², and more preferably at least 7 kp/cm². Here, “strength” is the fracture force, also known as the tablet “hardness,” required to fracture a tablet formed from the materials, divided by the maximum cross-sectional area of the tablet normal to that force. The fracture force may be measured using a Schleuniger Tablet Hardness Tester, Model 6D. The compression force required to achieve this strength will depend on the size of the tablet, but generally will be greater than about 5 kp. Friability is a well-known measure of a dosage form's resistance to surface abrasion that measures weight loss in percentage after subjecting the dosage form to a standardized agitation procedure. Friability values of from 0.8 to 1.0% are regarded as constituting the upper limit of acceptability. Dosage forms having a strength of greater than about 5 kp/cm² generally are very robust, having a friability of less than about 0.5%.

Other methods for forming matrix sustained-release dosage forms are well known in the pharmaceutical arts. See for example, Remington: The Science and Practice of Pharmacy, 20^{th} Edition, 2000.

### Osmotic Sustained Release Dosage Forms

Alternatively, ziprasidone may be incorporated into an osmotic sustained release dosage form. Such dosage forms have at least two components: (a) the core which contains an osmotic agent and ziprasidone; and (b) a water permeable, non-dissolving and non-eroding coating surrounding the core, the coating controlling the influx of water to the core from an aqueous environment of use so as to cause drug release by extrusion of some or all of the core to the environment of use. The osmotic agent contained in the core of this dosage form may be an aqueous-swellable hydrophilic polymer or it may be an osmogen, also known as an osmagent. The coating is preferably polymeric, aqueous-permeable, and has at least one delivery port which is pre-formed or formed in situ. Examples of such dosage forms are well known in the art. See, for example, Remington: The Science and Practice of Pharmacy, 20^{th} Edition, 2000. Examples of such dosage forms are also disclosed in U.S. Pat. No. 6,706,283, the relevant disclosure of which is herein incorporated by reference.

In addition to ziprasidone, the core of the osmotic dosage form optionally includes an “osmotic agent.” By “osmotic agent” is meant any agent that creates a driving force for transport of water from the environment of use into the core of the dosage form. Exemplary osmotic agents are water-swellable hydrophilic polymers, and osmogens (or osmagens). Thus, the core may include water-swellable hydrophilic polymers, both ionic and nonionic, often referred to as “osmopolymers” and “hydrogels.” The amount of water-swellable hydrophilic polymers present in the core may range from about 5 to about 80 wt %, preferably 10 to 50 wt %. Exemplary materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolate. Other materials include hydrogels comprising interpenetrating networks of polymers that may be formed by addition or by condensation polymerization, the components of which may comprise hydrophilic and hydrophobic monomers such as those just mentioned. Preferred polymers for use as the water-swellable hydrophilic polymers include PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt %, preferably 10 to 50 wt %. Typical classes of suitable osmogens are water-soluble organic acids, salts and sugars that are capable of imbibing water to thereby effect an osmotic pressure gradient across the barrier of the surrounding coating. Typical useful osmogens include magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. Particularly preferred osmogens are glucose, lactose, sucrose, mannitol, xylitol and sodium chloride.

The core may include a wide variety of additives and excipients that enhance the performance of the dosage form or that promote stability, tableting or processing. Such additives and excipients include tableting aids, surfactants, water-soluble polymers, pH modifiers, fillers, binders, pigments, disintegrants, antioxidants, lubricants and flavorants. Exemplary of such components are microcrystalline cellulose; metallic salts of acids such as aluminum stearate, calcium stearate, magnesium stearate, sodium stearate, and zinc stearate; pH control agents such as buffers, organic acids and organic acid salts and organic and inorganic bases; fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmiticstearic) ester, sorbitan esters, such as sorbitan monostearate, saccharose monostearate, saccharose monopalmitate, and sodium stearyl fumarate; polyoxyethylene sorbitan esters; surfactants, such as alkyl sulfates such as sodium lauryl sulfate and magnesium lauryl sulfate; polymers such as polyethylene glycols, polyoxyethylene glycols, polyoxyethylene and polyoxypropylene ethers and their copolymers, and polytetrafluoroethylene; and inorganic materials such as talc and dibasic calcium phosphate; cyclodextrins; sugars such as lactose and xylitol; and sodium starch glycolate. Examples of disintegrants are sodium starch glycolate (e.g., Explotab™), microcrystalline cellulose (e.g., Avicel™), microcrystalline silicified cellulose (e.g., ProSolv™), croscarmellose sodium (e.g., Ac-Di-Sol™).

One embodiment of an osmotic dosage form consists of one or more drug layers containing ziprasidone, and a sweller layer that comprises a water-swellable polymer, with a coating surrounding the drug layer and sweller layer. Each layer may contain other excipients such as tableting aids, osmagents, surfactants, water-soluble polymers and water-swellable polymers.

Such osmotic delivery dosage forms may be fabricated in various geometries including bilayer, wherein the core comprises a drug layer and a sweller layer adjacent to each other; trilayer, wherein the core comprises a sweller layer “sandwiched” between two drug layers; and concentric, wherein the core comprises a central sweller composition surrounded by the drug layer.

The coating of such a tablet comprises a membrane permeable to water but substantially impermeable to drug and excipients contained within. The coating contains one or more exit passageways or ports in communication with the drug-containing layer(s) for delivering the drug composition. The drug-containing layer(s) of the core contains the drug composition (including optional osmagents and hydrophilic water-soluble polymers), while the sweller layer consists of an expandable hydrogel, with or without additional osmotic agents.

When placed in an aqueous medium, the tablet imbibes water through the membrane, causing the composition to form a dispensable aqueous composition, and causing the hydrogel layer to expand and push against the drug-containing composition, forcing the composition out of the exit passageway. The composition can swell, aiding in forcing the drug out of the passageway. Drug can be delivered from this type of delivery system either dissolved or dispersed in the composition that is expelled from the exit passageway.

The rate of drug delivery is controlled by such factors as the permeability and thickness of the coating, the osmotic pressure of the drug-containing layer, the degree of hydrophilicity of the hydrogel layer, and the surface area of the dosage form. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the dosage form's surface area.

Exemplary materials useful in forming the drug-containing composition, in addition to ziprasidone, include HPMC, PEO and PVP and other pharmaceutically acceptable carriers. In addition, osmagents such as sugars or salts, especially sucrose, lactose, xylitol, mannitol, or sodium chloride, may be added. Materials which are useful for forming the hydrogel layer include sodium CMC, PEO, poly (acrylic acid), sodium (polyacrylate), sodium croscarmellose, sodium starch glycolate, PVP, crosslinked PVP, and other high molecular weight hydrophilic materials. Particularly useful are PEO polymers having an average molecular weight from about 5,000,000 to about 7,500,000 daltons.

In the case of a bilayer geometry, the delivery port(s) or exit passageway(s) may be located on the side of the tablet containing the drug composition or may be on both sides of the tablet or even on the edge of the tablet so as to connect both the drug layer and the sweller layer with the exterior of the dosage form. The exit passageway(s) may be produced by mechanical means or by laser drilling, or by creating a difficult-to-coat region on the tablet by use of special tooling during tablet compression or by other means.

The osmotic dosage form can also be made with a homogeneous core surrounded by a semipermeable membrane coating, as in U.S. Pat. No. 3,845,770. Ziprasidone can be incorporated into a tablet core and a semipermeable membrane coating can be applied via conventional tablet-coating techniques such as using a pan coater. A drug delivery passageway can then be formed in this coating by drilling a hole in the coating, either by use of a laser or mechanical means. Alternatively, the passageway may be formed by rupturing a portion of the coating or by creating a region on the tablet that is difficult to coat, as described above.

A particularly useful embodiment of an osmotic dosage form comprises: (a) a single-layer compressed core comprising: (i) ziprasidone, (ii) a hydroxyethylcellulose, and (iii) an osmagent, wherein the hydroxyethylcellulose is present in the core from about 2.0% to about 35% by weight and the osmagent is present from about 15% to about 70% by weight; (b) a water-permeable and drug-impermeable layer surrounding the core; and (c) at least one passageway within the layer (b) for delivering the drug to a fluid environment surrounding the tablet. In a preferred embodiment, the dosage form is shaped such that the surface area to volume ratio (of a water-swollen tablet) is greater than 0.6 mm⁻¹; more preferably greater than 1.0 mm⁻¹. It is preferred that the passageway connecting the core with the fluid environment be situated along the tablet band area. A particularly preferred shape is an oblong shape where the ratio of the tablet tooling axes, i.e., the major and minor axes which define the shape of the tablet, are between 1.3 and 3; more preferably between 1.5 and 2.5. In one embodiment, the combination of ziprasidone and the osmagent have an average ductility from about 100 to about 200 MPa, an average tensile strength from about 0.8 to about 2.0 MPa, and an average brittle fracture index less than about 0.2. The single-layer core may optionally include a disintegrant, a bioavailability enhancing additive, and/or a pharmaceutically acceptable excipient, carrier or diluent. Such dosage forms are disclosed more fully in commonly owned, pending U.S. patent application Ser. No. 10/352,283, entitled “Osmotic Delivery System,” the disclosure of which are incorporated herein by reference.

Entrainment of particles of ziprasidone in the extruded fluid during operation of such osmotic dosage form is highly desirable. For the particles to be well entrained, the drug form is preferably well dispersed in the fluid before the particles have an opportunity to settle in the tablet core. One means of accomplishing this is by adding a disintegrant that serves to break up the compressed core into its particulate components. Examples of standard disintegrants included materials such as sodium starch glycolate (e.g., Explotab™ CLV), microcrystalline cellulose (e.g., Avicel™), microcrystalline silicified cellulose (e.g., ProSolv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the dosage form. Non-gelling, non-swelling disintegrants provide a more rapid dispersion of the drug particles within the core as water enters the core. Preferred non-gelling, non-swelling disintegrants are resins, preferably ion-exchange resins. A preferred resin is Amberlite™ IRP 88 (available from Rohm and Haas, Philadelphia, Pa.). When used, the disintegrant is present in amounts ranging from about 1-25% of the core composition.

Water-soluble polymers are added to keep particles of the drug suspended inside the dosage form before they can be delivered through the passageway(s) (e.g., an orifice). High viscosity polymers are useful in preventing settling. However, the polymer in combination with the drug is extruded through the passageway(s) under relatively low pressures. At a given extrusion pressure, the extrusion rate typically slows with increased viscosity. Certain polymers in combination with particles of the drug form high viscosity solutions with water but are still capable of being extruded from the tablets with a relatively low force. In contrast, polymers having a low weight-average, molecular weight (<about 300,000) do not form sufficiently viscous solutions inside the tablet core to allow complete delivery due to particle settling. Settling of the particles is a problem when such dosage forms are prepared with no polymer added, which leads to poor drug delivery unless the tablet is constantly agitated to keep the particles from settling inside the core. Settling is also problematic when the particles are large and/or of high density such that the rate of settling increases.

Preferred water-soluble polymers for such osmotic dosage forms do not interact with the drug. Non-ionic polymers are preferred. An example of a non-ionic polymer forming solutions having a high viscosity yet still extrudable at low pressures is Natrosol™ 250H (high molecular weight hydroxyethylcellulose, available from Hercules Incorporated, Aqualon Division, Wilmington, Del.; MW equal to about 1 million daltons and a degree of polymerization equal to about 3,700). Natrosol™ 250H provides effective drug delivery at concentrations as low as about 3% by weight of the core when combined with an osmagent. Natrosol™ 250H NF is a high-viscosity grade nonionic cellulose ether that is soluble in hot or cold water. The viscosity of a 1% solution of Natrosol™ 250H using a Brookfield LVT (30 rpm) at 25° C. is between about 1,500 and about 2,500 cps.

Preferred hydroxyethylcellulose polymers for use in these monolayer osmotic tablets have a weight-average, molecular weight from about 300,000 to about 1.5 million. The hydroxyethylcellulose polymer is typically present in the core in an amount from about 2.0% to about 35% by weight.

Another example of an osmotic dosage form is an osmotic capsule. The capsule shell or portion of the capsule shell can be semipermeable. The capsule can be filled either by a powder or liquid consisting of ziprasidone, excipients that imbibe water to provide osmotic potential, and/or a water-swellable polymer, or optionally solubilizing excipients. The capsule core can also be made such that it has a bilayer or multilayer composition analogous to the bilayer, trilayer or concentric geometries described above.

Another class of osmotic dosage form useful in this invention comprises coated swellable tablets, as described in EP 378 404, incorporated herein by reference. Coated swellable tablets comprise a tablet core comprising the solubility-improved form of the drug and a swelling material, preferably a hydrophilic polymer, coated with a membrane, which contains holes, or pores through which, in the aqueous use environment, the hydrophilic polymer can extrude and carry out the drug composition. Alternatively, the membrane may contain polymeric or low molecular weight water-soluble “porosigens”. Porosigens dissolve in the aqueous use environment, providing pores through which the hydrophilic polymer and drug may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser, mechanical, or other means. In this class of osmotic dosage forms, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release dosage forms may also be multilayered, as described in EP 378 404 A2.

The osmotic sustained release dosage forms of the present invention also comprise a coating. The essential constraints on the coating for an osmotic dosage form are that it be water-permeable, have at least one port for the delivery of drug, and be non-dissolving and non-eroding during release of the drug formulation, such that drug is substantially entirely delivered through the delivery port(s) or pores as opposed to delivery primarily via permeation through the coating material itself. By “delivery port” is meant any passageway, opening or pore whether made mechanically, by laser drilling, by pore formation either during the coating process or in situ during use or by rupture during use. The coating should be present in an amount ranging from about 5 to 30 wt %, preferably 10 to 20 wt % relative to the core weight.

A preferred form of coating is a semipermeable polymeric membrane that has the port(s) formed therein either prior to or during use. Thickness of such a polymeric membrane may vary between about 20 and 800 μm, and is preferably in the range of 100 to 500 μm. The delivery port(s) should generally range in size from 0.1 to 3000 μm or greater, preferably on the order of 50 to 3000 μm in diameter. Such port(s) may be formed post-coating by mechanical or laser drilling or may be formed in situ by rupture of the coatings; such rupture may be controlled by intentionally incorporating a relatively small weak portion into the coating. Delivery ports may also be formed in situ by erosion of a plug of water-soluble material or by rupture of a thinner portion of the coating over an indentation in the core. In addition, delivery ports may be formed during coating, as in the case of asymmetric membrane coatings of the type disclosed in U.S. Pat. Nos. 5,612,059 and 5,698,220, the disclosures of which are incorporated by reference.

When the delivery port is formed in situ by rupture of the coating, a particularly preferred embodiment is a collection of beads that may be of essentially identical or of a variable composition. Drug is primarily released from such beads following rupture of the coating and, following rupture, such release may be gradual or relatively sudden. When the collection of beads has a variable composition, the composition may be chosen such that the beads rupture at various times following administration, resulting in the overall release of drug being sustained for a desired duration.

Coatings may be dense, microporous or “asymmetric,” having a dense region supported by a thick porous region such as those disclosed in U.S. Pat. Nos. 5,612,059 and 5,698,220. When the coating is dense the coating is composed of a water-permeable material. When the coating is porous, it may be composed of either a water-permeable or a water-impermeable material. When the coating is composed of a porous water-impermeable material, water permeates through the pores of the coating as either a liquid or a vapor.

Examples of osmotic dosage forms that utilize dense coatings include U.S. Pat. Nos. 3,995,631 and 3,845,770, the disclosures of which pertaining to dense coatings are incorporated herein by reference. Such dense coatings are permeable to the external fluid such as water and may be composed of any of the materials mentioned in these patents as well as other water-permeable polymers known in the art.

The membranes may also be porous as disclosed in U.S. Pat. Nos. 5,654,005 and 5,458,887 or even be formed from water-resistant polymers. U.S. Pat. No. 5,120,548 describes another suitable process for forming coatings from a mixture of a water-insoluble polymer and a leachable water-soluble additive, the pertinent disclosures of which are incorporated herein by reference. The porous membranes may also be formed by the addition of pore-formers as disclosed in U.S. Pat. No. 4,612,008, the pertinent disclosures of which are incorporated herein by reference.

In addition, vapor-permeable coatings may even be formed from extremely hydrophobic materials such as polyethylene or polyvinylidene difluoride that, when dense, are essentially water-impermeable, as long as such coatings are porous.

Materials useful in forming the coating include various grades of acrylics, vinyls, ethers, polyamides, polyesters and cellulosic derivatives that are water-permeable and water-insoluble at physiologically relevant pHs, or are susceptible to being rendered water-insoluble by chemical alteration such as by crosslinking.

Specific examples of suitable polymers (or crosslinked versions) useful in forming the coating include plasticized, unplasticized and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, and ethyl cellulose, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes.

A preferred coating composition comprises a cellulosic polymer, in particular cellulose ethers, cellulose esters and cellulose ester-ethers, i.e., cellulosic derivatives having a mixture of ester and ether substituents.

Another preferred class of coating materials are poly(acrylic) acids and esters, poly(methacrylic) acids and esters, and copolymers thereof.

A more preferred coating composition comprises cellulose acetate. An even more preferred coating comprises a cellulosic polymer and PEG. A most preferred coating comprises cellulose acetate and PEG.

Coating is conducted in conventional fashion, typically by dissolving or suspending the coating material in a solvent and then coating by dipping, spray coating or preferably by pan-coating. A preferred coating solution contains 5 to 15 wt % polymer. Typical solvents useful with the cellulosic polymers mentioned above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, water, and mixtures thereof. Pore-formers and non-solvents (such as water, glycerol and ethanol) or plasticizers (such as diethyl phthalate) may also be added in any amount as long as the polymer remains soluble at the spray temperature. Pore-formers and their use in fabricating coatings are described in U.S. Pat. No. 5,612,059, the pertinent disclosures of which are incorporated herein by reference.

Coatings may also be hydrophobic microporous layers wherein the pores are substantially filled with a gas and are not wetted by the aqueous medium but are permeable to water vapor, as disclosed in U.S. Pat. No. 5,798,119, the pertinent disclosures of which are incorporated herein by reference. Such hydrophobic but water-vapor permeable coatings are typically composed of hydrophobic polymers such as polyalkenes, polyacrylic acid derivatives, polyethers, polysulfones, polyethersulfones, polystyrenes, polyvinyl halides, polyvinyl esters and ethers, natural waxes and synthetic waxes. Especially preferred hydrophobic microporous coating materials include polystyrene, polysulfones, polyethersulfones, polyethylene, polypropylene, polyvinyl chloride, polyvinylidene fluoride and polytetrafluoroethylene. Such hydrophobic coatings can be made by known phase inversion methods using any of vapor-quench, liquid quench, thermal processes, leaching soluble material from the coating or by sintering coating particles. In thermal processes, a solution of polymer in a latent solvent is brought to liquid-liquid phase separation in a cooling step. When evaporation of the solvent is not prevented, the resulting membrane will typically be porous. Such coating processes may be conducted by the processes disclosed in U.S. Pat. Nos. 4,247,498; 4,490,431 and 4,744,906, the disclosures of which are also incorporated herein by reference.

Osmotic sustained-release dosage forms may be prepared using procedures known in the pharmaceutical arts. See for example, Remington: The Science and Practice of Pharmacy, 20^{th} Edition, 2000.

### Multiparticulates

The dosage forms of the present invention may also provide sustained release of ziprasidone through the use of multiparticulates. Multiparticulates generally refer to dosage forms that comprise a multiplicity of particles or granules that may range in size from about 10 μm to about 2 mm, more typically about 50 μm to 1 mm in diameter. Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art.

Such multiparticulates may be made by any known process, such as wet- and dry-granulation processes, extrusion/spheronization, roller-compaction, melt-congealing, or by spray-coating seed cores. For example, in wet- and dry-granulation processes, the composition comprising ziprasidone and optional excipients may be granulated to form multiparticulates of the desired size. Other excipients, such as a binder (e.g., microcrystalline cellulose), may be blended with the composition to aid in processing and forming the multiparticulates. In the case of wet granulation, a binder such as microcrystalline cellulose may be included in the granulation fluid to aid in forming a suitable multiparticulate. See, for example, Remington: The Science and Practice of Pharmacy, 20^{th} Edition, 2000.

In any case, the resulting particles may themselves constitute the multiparticulate dosage form or they may be coated by various film-forming materials such as enteric polymers or water-swellable or water-soluble polymers, or they may be combined with other excipients or vehicles to aid in dosing to patients.

### Enteric Coated Cores

The sustained release means may comprise a core coated with an enteric coating so that the core does not dissolve in the stomach. The core may be either a sustained release core, such as a matrix tablet or an osmotic tablet, or alternatively may be an immediate release core that provides a delayed burst. By “enteric coating” is meant an acid resistant coating that remains intact and does not dissolve at pH of less than about 4. The enteric coating surrounds the core so that the core does not dissolve in the stomach. The enteric coating may include an enteric coating polymer. Enteric coating polymers are generally polyacids having a pKₐ of about 3 to 5. Examples of enteric coating polymers include: cellulose derivatives, such as cellulose acetate phthalate, cellulose acetate trimellitate, hydroxypropyl methyl cellulose acetate succinate, cellulose acetate succinate, carboxy methyl ethyl cellulose, methylcellulose phthalate, and ethylhydroxy cellulose phthalate; vinyl polymers, such as polyvinyl acetate phthalate, polyvinylbutyrate acetate, vinyl acetate-maleic anhydride copolymer; polyacrylates; and polymethacrylates such as methyl acrylate-methacrylic acid copolymer, methacrylate-methacrylic acid-octyl acrylate copolymer; and styrene-maleic mono-ester copolymer. These may be used either alone or in combination, or together with other polymers than those mentioned above.

One class of preferred coating materials are the pharmaceutically acceptable methacrylic acid copolymer which are copolymers, anionic in character, based on methacrylic acid and methyl methacrylate, for example having a ratio of free carboxyl groups:methyl-esterified carboxyl groups of 1:>3, e.g. around 1:1 or 1:2, and with a mean molecular weight of 135000. Some of these polymers are known and sold as enteric polymers, for example having a solubility in aqueous media at pH 5.5 and above, such as the commercially available EUDRAGIT enteric polymers, such as Eudragit L 30, a cationic polymer synthesized from dimethylaminoethyl methacrylate, Eudragit S and Eudragit NE.

The coating may include conventional plasticizers, including dibutyl phthalate; dibutyl sebacate; diethyl phthalate; dimethyl phthalate; triethyl citrate; benzyl benzoate; butyl and glycol esters of fatty acids; mineral oil; oleic acid; stearic acid; cetyl alcohol; stearyl alcohol; castor oil; corn oil; coconut oil; and camphor oil; and other excipients such as anti-tack agents, glidants, etc. For plasticizers, triethyl citrate, coconut oil and dibutyl sebacate are particularly preferred. Typically the coating may include from about 0.1 to about 25 wt. % plasticizer and from about 0.1 to about 10 wt % anti-tack agent.

The enteric coating may also include insoluble materials, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin, 1,2-, 3,4-diepoxybutane, etc. The enteric coating may also include starch and/or dextrin.

The enteric coating may be applied to the core by dissolving or suspending the enteric coating materials in a suitable solvent. Examples of solvents suitable for use in applying a coating include alcohols, such as methanol, ethanol, isomers of propanol and isomers of butanol; ketones, such as acetone, methylethyl ketone and methyl isobutyl ketone; hydrocarbons, such as pentane, hexane, heptane, cyclohexane, methylcyclohexane, and octane; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; acetonitrile; water; and mixtures thereof.

Coating may be conducted by conventional techniques, such as by pan coaters, rotary granulators and fluidized bed coaters such as top-spray, tangential-spray or bottom-spray (Würster coating), most preferably the latter.

One preferred coating solution consists of about 40 wt % Eudragit L30-D55 and 2.5 wt % triethylcitrate in about 57.5 wt % water. This enteric coating solution may be coated onto the core using a pan coater.

### Immediate Release

While the sustained release oral dosage forms release at least a portion of the ziprasidone after 2 hours after administration to the use environment, the sustained release dosage may also have an immediate release portion. By “immediate release portion” is meant broadly that a portion of the ziprasidone separate from the sustained release means is released within the two hours or less following administration to a gastric use environment. “Administration” to a use environment means, where the in vivo use environment is the GI tract, delivery by ingestion or swallowing or other such means to deliver the dosage form. Where the use environment is in vitro, “administration” refers to placement or delivery of the dosage form to the in vitro test medium. The dosage form may release at least 70 wt % of the ziprasidone initially present in the immediate release portion of the dosage form within two hours or less following introduction to a gastric use environment. Preferably, the dosage form releases at least 80 wt % during the first two hours, and most preferably, at least 90 wt % of the drug initially in the immediate release portion of the dosage form during the first two hours after administering of the dosage form to a gastric use environment. Immediate release of drug may be accomplished by any means known in the pharmaceutical arts, including immediate release coatings, immediate release layers, and immediate release multiparticulates or granules.

Virtually any means for providing immediate release of a drug known in the pharmaceutical arts can be used with the dosage form of the present invention. In one embodiment, the ziprasidone in the immediate release portion is in the form of an immediate release coating that surrounds the sustained release means. The drug in the immediate release portion may be combined with a water soluble or water dispersible polymer, such as HPC, HPMC, HEC, PVP, and the like. The coating can be formed using solvent-based coating processes, powder-coating processes, and hot-melt coating processes, all well known in the art. In solvent-based processes, the coating is made by first forming a solution or suspension comprising the solvent, the drug, the coating polymer and optional coating additives. Preferably, the drug is suspended in the coating solvent. The coating materials may be completely dissolved in the coating solvent, or only dispersed in the solvent as an emulsion or suspension or anywhere in between. Latex dispersions, including aqueous latex dispersions, are a specific example of an emulsion or suspension that may be useful as a coating solution. The solvent used for the solution should be inert in the sense that it does not react with or degrade the drug, and be pharmaceutically acceptable. In one aspect, the solvent is a liquid at room temperature. Preferably, the solvent is a volatile solvent. By “volatile solvent” is meant that the material has a boiling point of less than about 150° C. at ambient pressure, although small amounts of solvents with higher boiling points can be used and acceptable results still obtained.

Examples of solvents suitable for use in applying a coating to an enteric coated sustained release core include alcohols, such as methanol, ethanol, isomers of propanol and isomers of butanol; ketones, such as acetone, methylethyl ketone and methyl isobutyl ketone; hydrocarbons, such as pentane, hexane, heptane, cyclohexane, methylcyclohexane, octane and mineral oil; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; acetonitrile; water; and mixtures thereof.

The coating formulation may also include additives to promote the desired immediate release characteristics or to ease the application or improve the durability or stability of the coating. Types of additives include plasticizers, pore formers, and glidants. Examples of coating additives suitable for use in the compositions of the present invention include plasticizers, such as mineral oils, petrolatum, lanolin alcohols, polyethylene glycol, polypropylene glycol, triethyl citrate, sorbitol, triethanol amine, diethyl phthalate, dibutyl phthalate, castor oil, triacetin and others known in the art; emulsifiers, such as polysorbate-80; pore formers, such as polyethylene glycol, polyvinyl pyrrolidone, polyethylene oxide, hydroxyethyl cellulose and hydroxypropylmethyl cellulose; and glidants, such as colloidal silicon dioxide, talc and cornstarch. In one embodiment, the drug is suspended in a commercially available coating formulation, such as Opadry® clear (available from Colorcon, Inc., WestPoint, Pa.). Coating is conducted in conventional fashion, typically by dipping, fluid-bed coating, spray-coating, or pan-coating.

The immediate release coating may also be applied using powder coating techniques well known in the art. In these techniques, the drug is blended with optional coating excipients and additives, to form an immediate release coating composition. This composition may then be applied using compression forces, such as in a tablet press.

The coating may also be applied using a hot-melt coating technique. In this method, a molten mixture comprising the drug and optional coating excipients and additives, is formed and then sprayed onto the enteric coated sustained release core. Typically, the hot-melt coating is applied in a fluidized bed equipped with a top-spray arrangement.

In another embodiment, the immediate release portion is first formed into an immediate release composition, multiparticulates or granules that are combined with the sustained release means. The immediate release composition, multiparticulates, or granules may be combined with the sustained release means in a capsule. In one aspect, the immediate-release composition consists essentially of the drug. In another aspect, the immediate-release composition comprises ziprasidone and optional excipients, such as binders, stabilizing agents, diluents, disintegrants, and surfactants. Such immediate release compositions may be formed by any conventional method for combining the drug and excipients. Exemplary methods include wet and dry granulation. In another embodiment, immediate release multiparticulates are filled into the same gelatin capsule as the sustained release multiparticulates, or, the immediate release multiparticulates are blended with the sustained release multiparticulates along with other excipients and compressed into tablets.

In addition to the drug, the immediate release portion may include other excipients to aid in formulating the immediate release portion. See, for example, Remington: The Science and Practice of Pharmacy (20th ed. 2000). Examples of other excipients include disintegrants, porosigens, matrix materials, fillers, diluents, lubricants, glidants, and the like, such as those previously described.

The relative amount of ziprasidone in the immediate release portion and the sustained release portion may be as desired in order to obtain desired blood levels of drug. The immediate release portion may contain at least 10 wt %, at least 20 wt %, or even at least 30 wt % of the ziprasidone in the dosage form. In exemplary embodiments, the immediate release portion may contain from about 10 to 50 wt % of the ziprasidone, while the sustained release means may contain from about 90 wt % to about 50 wt % of the ziprasidone.

### Dosage Form Excipients

The sustained release dosage form may contain other excipients to improve performance, handling, or processing. Generally, excipients such as surfactants, pH modifiers, fillers, matrix materials, complexing agents, solubilizers, pigments, lubricants, glidants, flavorants, and so forth may be used for customary purposes and in typical amounts without adversely affecting the properties of the sustained release dosage form. See for example, Remington's Pharmaceutical Sciences (18th ed. 1990).

One very useful class of excipients is surfactants, preferably present from 0 to 10 wt %. Suitable surfactants include fatty acid and alkyl sulfonates; commercial surfactants such as benzalkonium chloride (HYAMINE® 1622, available from Lonza, Inc., Fairlawn, N.J.); dioctyl sodium sulfosuccinate (DOCUSATE SODIUM, available from Mallinckrodt Spec. Chem., St. Louis, Mo.); polyoxyethylene sorbitan fatty acid esters (TWEEN®, available from ICI Americas Inc., Wilmington, Del.; LIPOSORB® O-20, available from Lipochem Inc., Patterson N.J.; CAPMUL® POE-0, available from Abitec Corp., Janesville, Wis.); and natural surfactants such as sodium taurocholic acid, 1-palmitoyl-2-oleoyl-sn-glycero-3-phosphocholine, lecithin, and other phospholipids and mono- and diglycerides. Such materials can advantageously be employed to increase the rate of dissolution by, for example, facilitating wetting, or otherwise increase the rate of drug release from the dosage form.

The addition of pH modifiers such as acids, bases, or buffers may be beneficial, retarding the dissolution of ziprasidone (e.g., bases such as sodium acetate or amines) or, alternatively, enhancing the rate of dissolution of ziprasidone (e.g., acids such as citric acid or succinic acid).

Conventional matrix materials, complexing agents, solubilizers, fillers, disintegrating agents (disintegrants), or binders may also comprise up to 90 wt % of the dosage form.

Examples of fillers, or diluents include lactose, mannitol, xylitol, microcrystalline cellulose, dibasic calcium phosphate (anhydrous and dihydrate) and starch.

Examples of disintegrants include sodium starch glycolate, sodium alginate, carboxy methyl cellulose sodium, methyl cellulose, and croscarmellose sodium, and crosslinked forms of polyvinyl pyrrolidone such as those sold under the trade name CROSPOVIDONE (available from BASF Corporation).

Examples of binders include methyl cellulose, microcrystalline cellulose, starch, and gums such as guar gum, and tragacanth.

Examples of lubricants include magnesium stearate, calcium stearate, and stearic acid.

Examples of preservatives include sulfites (an antioxidant), benzalkonium chloride, methyl paraben, propyl paraben, benzyl alcohol and sodium benzoate.

Examples of suspending agents or thickeners include xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, and titanium dioxide.

Examples of anti-caking agents or fillers include silicon oxide and lactose.

Examples of solubilizers include ethanol, propylene glycol or polyethylene glycol.

Other conventional excipients may be employed in the sustained release dosage forms of this invention, including those well-known in the art. Generally, excipients such as pigments, lubricants, flavorants, and so forth may be used for customary purposes and in typical amounts without adversely affecting the properties of the compositions.

### Dosing Interval

The sustained release dosage forms may be administered at any convenient frequency. In one embodiment, the sustained release dosage forms are administered at least twice per day. In one embodiment, the dosage forms are administered twice per day. When dosed twice per day, the period between dosing is preferably from 8 to 16 hours. The dosage forms are preferably administered with food. For example, when the dosage forms are administered twice per day, a dosage form may be administered in the morning with a meal, and another dosage form of the same composition may be administered again in the evening with a meal.

In one embodiment, the sustained release means provide a relatively short release period that may be suitable for twice daily administration. The release period for such dosage forms may be from 4 to 8 hours. By “release period” is meant the time required for the dosage form to release 80 wt % of the ziprasidone in the dosage form. The amount of drug in the dosage from may be 20 mgA, 30 mgA, 40 mgA, 60 mgA, 80 mgA, or more. In a preferred embodiment, ziprasidone in such a short release dosage form is preferably a high solubility salt form of ziprasidone. The dosage form is preferably administered twice a day in the fed state.

In another embodiment, the sustained release dosage form is administered only once per day. The dosage forms are preferably administered with food. Accordingly, when a dosage form is administered once per day, the dosage form may be administered once in the morning with a meal, or may be administered once in the evening with a meal.

In another embodiment, the sustained release means provide a relatively long release period that may be suitable for twice daily administration. The release period for such dosage forms may be from 8 to 24 hours. By “release period” is meant the time required for the dosage form to release 80 wt % of the ziprasidone in the dosage form. The amount of drug in the dosage from may be 20 mgA, 30 mgA, 40 mgA, 60 mgA, 80 mgA, or more. In a preferred embodiment, ziprasidone in such a short release dosage form is in a solubility-improved form of ziprasidone and contains a precipitation inhibiting polymer. The dosage form is preferably administered once a day in the fed state.

The sustained release dosage forms may be used to treat any condition for which ziprasidone may be effective.

Other features and embodiments of the invention will become apparent from the following examples that are given for illustration of the invention rather than for limiting its intended scope.

### EXAMPLES

### Solubility-Improved Forms of Ziprasidone High Solubility Salt Forms

Microcentrifuge dissolution tests were performed to evaluate the hydrochloride and mesylate crystalline salt forms of ziprasidone to verify they were solubility-improved forms of ziprasidone. For this test, a sufficient amount of ziprasidone hydrochloride monohydrate or ziprasidone mesylate trihydrate was added to a microcentrifuge test tube so that the concentration of ziprasidone would have been 200 μgA/mL, if all of the ziprasidone had dissolved. The tests were run in duplicate. The tubes were placed in a 37° C. temperature-controlled chamber, and 1.8 mL MFD solution at pH 6.5 and 290 mOsm/kg was added to each respective tube. The samples were quickly mixed using a vortex mixer for about 60 seconds. The samples were centrifuged at 13,000 G at 37° C. for 1 minute prior to collecting a sample. The resulting supernatant solution was then sampled and diluted 1:5 (by volume) with methanol. Samples were analyzed by high-performance liquid chromatography (HPLC) at a UV absorbance of 315 nm using a Zorbax RxC8 Reliance column and a mobile phase consisting of 55% (50 mM potassium dihydrogen phosphate, pH 6.5)/45% acetonitrile. Drug concentration was calculated by comparing UV absorbance of samples to the absorbance of drug standards. The contents of each tube were mixed on the vortex mixer and allowed to stand undisturbed at 37° C. until the next sample was taken. Samples were collected at 4, 10, 20, 40, 90, and 1200 minutes following administration to the MFD solution. The results are shown in Table 1.

A similar test was performed with the crystalline ziprasidone free base as a control, and a sufficient amount of material was added so that the concentration of compound would have been 200 μgA/mL, if all of the ziprasidone had dissolved.

TABLE 1

| | Time | Dissolved Ziprasidone | AUC |
|---|---|---|---|
| Salt Form | (min) | Concentration (μgA/mL) | (min-μgA/mL) |
| Ziprasidone | 0 | 0 | 0 |
| Free Base | 4 | 1 | 3 |
| | 10 | 1 | 11 |
| | 20 | 1 | 23 |
| | 40 | 2 | 51 |
| | 90 | 1 | 120 |
| | 1200 | 2 | 2000 |
| Ziprasidone | 0 | 0 | 0 |
| hydrochloride | 4 | 14 | 30 |
| monohydrate | 10 | 15 | 110 |
| | 20 | 20 | 280 |
| | 40 | 22 | 700 |
| | 90 | 18 | 1,700 |
| | 1200 | 9 | 16,400 |
| Ziprasidone | 0 | 0 | 0 |
| mesylate | 4 | 55 | 110 |
| trihydrate | 10 | 33 | 380 |
| | 20 | 20 | 640 |
| | 40 | 13 | 970 |
| | 90 | 11 | 1,600 |
| | 1200 | 6 | 11,200 |

The concentrations of ziprasidone obtained in these tests were used to determine the maximum dissolved concentration of ziprasidone (“MDC₉₀”) and the area under the concentration-versus-time curve (“AUC₉₀”) during the initial ninety minutes. The results are shown in Table 2.

TABLE 2

| | MDC₉₀ | AUC₉₀ |
|---|---|---|
| Salt Form | (μgA/mL) | (min*μgA/mL) |
| Ziprasidone free base | 2 | 120 |
| Ziprasidone hydrochloride | 22 | 1,700 |
| monohydrate | | |
| Ziprasidone mesylate trihydrate | 55 | 1,600 |

These results show that ziprasidone hydrochloride monohydrate provided an MDC₉₀ that was 11-fold that provided by the free base, and an AUC₉₀ that was 14-fold that provided by the free base. The ziprasidone mesylate trihydrate provided an MDC₉₀ that was 27-fold that provided by the free base, and an AUC₉₀ that was 13-fold that provided by the free base. Thus, both the hydrochloride and mesylate salt forms are solubility-improved forms of ziprasidone.

### Ziprasidone Crystals Coated with Precipitation-Inhibiting Polymers

Ziprasidone coated crystals comprising 35% active ziprasidone hydrochloride monohydrate coated with the precipitation-inhibiting polymer HPMCAS, were prepared as follows. A spray suspension was first formed by dissolving HPMCAS-H (AQOAT H grade, available from Shin Etsu, Tokyo Japan) in acetone in a container equipped with a top-mounted mixer. Crystalline particles of ziprasidone hydrochloride monohydrate, having a mean particle size of about 10 μm, were then added to the polymer solution and mixing continued with a top-mounted mixer. The composition consisted of 3.97 wt % crystalline ziprasidone hydrochloride monohydrate particles suspended in 6.03 wt % HPMCAS-HG, and 90 wt % acetone. Next, a re-circulation pump (Yamada air actuated diaphragm pump model NDP-5FST) was used to transfer the suspension to a high-shear in line mixer (Bematek model LZ-150-6-PB multi-shear in-line mixer) where a series of rotor/stator shear heads broke up any remaining drug crystal agglomerations. The high shear mixer was operated with a setting of 3500±500 rpm, for 45-60 minutes per 20 kg solution. The re-circulation pump pressure was 35±10 psig.

The suspension was then pumped using a high-pressure pump to a spray drier (a Niro type XP Portable Spray-Dryer with a Liquid-Feed Process Vessel (“PSD-1”)), equipped with a pressure nozzle (Spraying Systems Pressure Nozzle and Body—SK 74-20). The PSD-1 was equipped with a 5-foot 9-inch chamber extension. The chamber extension was added to the spray dryer to increase the vertical length of the dryer. The added length increased the residence time within the dryer, which allowed the product to dry before reaching the angled section of the spray dryer. The spray drier was also equipped with a 316 stainless steel circular diffuser plate with 1/16-inch drilled holes, having a 1% open area. This small open area directed the flow of the drying gas to minimize product recirculation within the spray dryer. The nozzle sat flush with the diffuser plate during operation. The suspension was delivered to the nozzle at about 285 g/min at a pressure of about 300 psig. The pump system included a pulsation dampener to minimize pulsation at the nozzle. Drying gas (e.g., nitrogen) was circulated through the diffuser plate at a flow rate of 1850 g/min, and an inlet temperature of 140° C. The evaporated solvent and wet drying gas exited the spray drier at a temperature of 40° C. The coated crystals formed by this process were collected in a cyclone, then post-dried using a Gruenberg single-pass convection tray dryer operating at 40° C. for 4 hours. The properties of the coated crystals after post-drying were as follows:

| Parameter | Value |
|---|---|
| Morphology | Irregular spheres with evidence |
| | of crystalline particles |
| Crystallinity (% of drug) | 90% ± 10% |
| Mean particle diameter (μm) | 42 |
| *Dv₁₀, Dv₅₀, Dv₉₀ (μm) | 13, 38, 76 |
| Span (D₉₀ − D₁₀)/D₅₀ | 1.6 |
| Bulk specific volume (cc/g) | 3.3 |
| Tapped specific volume (cc/g) | 2.2 |
| Hausner ratio | 1.5 |
| Glass Transition Temperature at | 120 (the same as the Tg |
| 5% RH (° C.) | for HPMCAS-HG) |
| Crystallization Temperature (° C.) | None Observed from |
| | 0° C. to 250° C. |

*10 vol % of the particles have a diameter that is smaller than D₁₀; 50 vol % of the particles have a diameter that is smaller than D₅₀, and 90 vol % of the particles have a diameter that is smaller than D₉₀.

The ziprasidone coated crystals were evaluated in vitro using a membrane permeation test. An Accurel® PP 1E microporous polypropylene membrane was obtained from Membrana GmbH (Wuppertal, Germany). The membrane was washed in isopropyl alcohol and rinsed in methanol in a sonicating bath for 1 minute at ambient temperature, and then allowed to air dry at ambient temperature. The feed side of the membrane was then plasma-treated to render it hydrophilic by placing a sample of the membrane in a plasma chamber. The atmosphere of the plasma chamber was saturated with water vapor at a pressure of 550 mtorr. A plasma was then generated using radio frequency (RF) power inductively coupled into the chamber via annular electrodes at a power setting of 50 watts for 45 seconds. The contact angle of a drop of water placed on the surface of the plasma-treated membrane was about 40°. The contact angle of a drop of water placed on the permeate side of the same membrane was greater than about 110°.

A permeate reservoir was formed by gluing a sample of the plasma-treated membrane to a glass tube having an inside diameter of about 1 inch (2.54 cm) using an epoxy-based glue (LOCTITE® E-30CL HYSOL® from Henkel Loctite Corp, Rocky Hill, Conn.). The feed-side of the membrane was oriented so that it was on the outside of the permeate reservoir, while the permeate-side of the membrane was oriented so that it was on the inside of the reservoir. The effective membrane area of the membrane on the permeate reservoir was about 4.9 cm². The permeate reservoir was placed into a glass feed reservoir. The feed reservoir was equipped with a magnetic stir bar and the reservoir was placed on a stir plate and the stir rate was set to 100 rpm during the test. The apparatus was placed into a chamber maintained at 37° C. for the duration of the test. Further details of the test apparatus and protocols are presented in co-pending U.S. Patent Application Ser. No. 60/557,897, entitled “Method and Device for Evaluation of Pharmaceutical Compositions,” filed Mar. 30, 2004 (attorney Docket No. PC25968), incorporated herein by reference.

To form the feed solution, a 1.39 mg sample of the coated crystals was weighed into the feed reservoir. To this was added 5 mL of MFD solution previously described, consisting of PBS solution containing 7.3 mM sodium taurocholic acid and 1.4 mM of 1-palmitoyl-2-oleyl-sn-glycero-3-phosphocholine (0.5% NaTC/POPC). The concentration of ziprasidone in the feed solution would have been 100 μgA/mL, if all of the ziprasidone had dissolved. The feed solution was mixed using a vortex mixer for 1 minute. Before the membrane contacted the feed solution, 5 mL of 60 wt % decanol in decane was placed into the permeate reservoir. Time zero in the test was when the membrane was placed in contact with the feed solution. A 50 mL aliquot of the permeate solution was collected at the times indicated. Samples were then diluted in 250 mL IPA and analyzed using HPLC. The results are shown in Table 3.

As a control, the membrane test was repeated using a 0.5-mg sample of crystalline ziprasidone alone, so that the concentration of drug would have been 100 μg/mL, if all of the drug had dissolved. These results are also given in Table 3.

TABLE 3

| Formulation | Time (min) | Concentration (μgA/mL) |
|---|---|---|
| Ziprasidone | 0 | 0.0 |
| Coated | 20 | 3.4 |
| Crystals | 40 | 13.2 |
| | 60 | 17.5 |
| | 90 | 25.2 |
| | 120 | 33.3 |
| | 180 | 47.9 |
| | 240 | 48.4 |
| | 360 | 52.4 |
| Crystalline | 0 | 0.0 |
| Ziprasidone | 20 | 5.2 |
| HCl | 40 | 8.1 |
| | 60 | 10.0 |
| | 90 | 11.4 |
| | 120 | 12.9 |
| | 180 | 18.1 |
| | 245 | 20.9 |
| | 360 | 22.6 |

The maximum flux of drug across the membrane (in units of mgA/cm²-min) was determined by performing a least-squares fit to the data in Table 3 from 0 to 60 minutes to obtain the slope, multiplying the slope by the permeate volume (5 mL), and dividing by the membrane area (4.9 cm²). The results of this analysis are summarized in Table 4, and show that the ziprasidone coated crystals provided a maximum flux through the membrane that was 2-fold that provided by crystalline ziprasidone free base alone.

TABLE 4

| | Maximum flux of Ziprasidone |
|---|---|
| Formulation | (mgA/cm²-min) |
| Ziprasidone Coated Crystals | 0.32 |
| Crystalline Ziprasidone HCl | 0.16 |

### Preparation of Sustained-Release Dosage Forms

### Dosage Form DF-1

A dosage form containing ziprasidone hydrochloride monohydrate was prepared that provided sustained-release of ziprasidone. The dosage form was in the form of a bi-layer osmotic tablet. The bi-layer osmotic tablet consisted of a drug-containing composition, a water-swellable composition, and a coating around the two layers. The bi-layer tablet was prepared as follows.

### Preparation of the Drug-Containing Composition

To form the drug-containing composition, the following materials were blended: 10.0 wt % ziprasidone hydrochloride monohydrate, 84.0 wt % polyethylene oxide (PEO) (Polyox WSR N80) having an average molecular weight of 200,000, 5.0 wt % hydroxypropyl cellulose, and 1.0 wt % magnesium stearate. The drug-containing composition ingredients were first combined without magnesium stearate, and wet-granulated using IPA/water (85/15) in a Niro SP1 high shear mixer granulator. The granulation was sieved wet, and then dried in a convection oven at 40° C. for 16 hours. The dried granulation was then milled using a Fitzpatrick M5A mill. Finally, the magnesium stearate was added to the drug-containing composition in a twin-shell blender, and the ingredients were blended for an additional 5 minutes.

### Preparation of the Water-Swellable Composition

To form the water-swellable composition, the following materials were blended: 64.9 wt % polyethylene oxide (Polyox WSR coagulant) having an average molecular weight of 5,000,000, 34.5 wt % sodium chloride, 0.5 wt % magnesium stearate, and 0.1 wt % Blue Lake #2. First, the PEO and sodium chloride were combined and blended in a twin shell blender for 10 minutes, then milled using a Fitzpatrick M5A mill. The Blue Lake #2 was sieved with a 40-mesh screen, and added to a portion of the PEO and sodium chloride. The ingredients were mixed using a Turbula mixer for 5 minutes, then added to the remaining PEO and sodium chloride, and blended in a twin-shell blender for 10 minutes. The magnesium stearate was added, and the mixture was blended again for 5 minutes.

### Preparation of Tablet Cores

Bilayer tablet cores were manufactured using an Elizabeth-Hata trilayer press combining 454.5 mg of the drug-containing composition and 150.5 mg of the water-swellable composition with 7/16-inch standard round concave (SRC) plain-faced tooling. The tablet cores were compressed to a hardness of about 12.6 kiloponds (kp). The resulting bi-layer tablet core had a total weight of 605 mg and contained a total of 40 mg active ziprasidone.

### Application of the Coating

Coatings for the tablet cores were applied in a Vector LDCS-30 pan coater. The coating solution for DF-1 contained cellulose acetate (CA 398-10 from Eastman Fine Chemical, Kingsport, Tenn.), polyethylene glycol (PEG 3350, Union Carbide), water, and acetone in a weight ratio of 7/3/5/85 (wt %). A Masterflex pump was used to deliver 20 g of solution per minute. The flow rate of the inlet heated drying gas of the pan coater was set at 40 ft³/min with the outlet temperature set at 28° C. Air at 22 psi was used to atomize the coating solution from the spray nozzle, with a nozzle-to-bed distance of 2⅝ inches. The pan rotation was set to 14 rpm. The so-coated tablets were dried 16 hr at 40° C. in a tray-drier. The final dry coating weight amounted to about 10 wt % of the tablet core. One 900 μm diameter hole was laser-drilled in the coating on the drug-containing composition side of each of the tablets of DF-1 to provide one delivery port per tablet.

### Dosage Form DF-2

Dosage Form DF-2 was prepared using the same procedure outlined for DF-1, except that for DF-2, the coating solution contained CA 398-10, PEG 3350, water, and acetone in a weight ratio of 8/2/5/85 (wt %).

### Dosage Form DF-3

A bilayer osmotic dosage form containing ziprasidone hydrochloride monohydrate was prepared using the following procedures.

### Preparation of the Drug-Containing Composition

To form the drug-containing composition, the following materials were blended: 10.0 wt % ziprasidone hydrochloride monohydrate, 84.0 wt % PEO (Polyox WSR N80), and 1.0 wt % magnesium stearate. The drug-containing composition ingredients were first combined without magnesium stearate, blended for 20 minutes in a Turbula mixer, passed through a 20 mesh sieve, and blended again for 20 minutes. One half of the magnesium stearate was then added to the blend and the mixture blended for an additional 4 minutes. Next, the ingredients were roller-compacted using a Vector TF mini roller-compactor (roller pressure 1 ton, roller speed 2 rpm, auger speed 1.0 rpm), then milled using a Fitzpatrick M5A mill equipped with a rasping screen at 1500 rpm. Finally, the remaining magnesium stearate was added, and the ingredients were blended again for 4 minutes.

### Preparation of the Water-Swellable Composition

To form the water-swellable composition, the following materials were blended: 65.0 wt % PEO (Polyox WSR coagulant), 34.3 wt % sodium chloride, 0.5 wt % magnesium stearate, and 0.2 wt % Blue Lake #2. All ingredients except magnesium stearate and Blue Lake #2 were combined and blended for 20 minutes, passed through a 20 mesh sieve, and blended again for 20 minutes. The magnesium stearate and Blue Lake #2 were then added, and the mixture was blended for 4 minutes.

### Preparation of Tablet Cores

Bilayer tablet cores were manufactured using an F press combining 444 mg of the drug-containing composition and 222 mg of the water-swellable composition with 15/32-inch standard round concave (SRC) plain-face tooling. The tablet cores were compressed to a hardness of about 9.1 kp. The resulting bi-layer tablet core had a total weight of 666 mg and contained a total of 40 mg active ziprasidone.

### Application of the Coating

Coatings for the tablet cores were applied in a Vector LDCS-20 pan coater. The coating solution contained CA 398-10, PEG 3350, water, and acetone in a weight ratio of 3.5/1.5/3/92 (wt %). The flow rate of the inlet heated drying gas of the pan coater was set at 40 ft³/min with the outlet temperature set at 25° C. Nitrogen at 20 psi was used to atomize the coating solution from the spray nozzle, with a nozzle-to-bed distance of 2 inches. The pan rotation was set to 20 rpm. The so-coated tablets were dried 16 hr at 40° C. in a tray-drier. The final dry coating weight amounted to about 16.4 wt % of the tablet core. One 900 μm diameter hole was laser-drilled in the coating on the drug-containing composition side of each of the tablets to provide one delivery port per tablet.

### Dosage Form DF-4

Dosage Form DF-4 was prepared using the same procedure outlined for DF-1 with the following exceptions. The drug-containing composition consisted of 11.96 wt % ziprasidone mesylate trihydrate, 82.04 wt % PEO (Polyox WSR N80), 5 wt % hydroxypropyl cellulose, and 1 wt % magnesium stearate. The water-swellable composition consisted of 65.0 wt % PEO (Polyox WSR Coagulant), 34.45 wt % sodium chloride, 0.5 wt % magnesium stearate, and 0.05 wt % Blue Lake #2. The coating solution contained CA 398-10, PEG 3350, water, and acetone in a weight ratio of 8/2/5/85 (wt %), and amounted to 10.4 wt % of the core weight. Each tablet of DF-4 contained 40 mgA of ziprasidone.

### Dosage Form DF-5

Dosage Form DF-5 was prepared using the same procedure outlined for DF-1 with the following exceptions. The drug-containing composition consisted of 7.7 wt % ziprasidone mesylate trihydrate, 31 wt % beta-cyclodextrin, 59.9 wt % PEO (Polyox WSR N80), 0.4 wt % hydroxypropyl methylcellulose acetate succinate (HPMCAS; the MF grade from Shin Etsu), and 1 wt % magnesium stearate. The water-swellable composition consisted of 65.0 wt % PEO (Polyox WSR Coagulant), 34.4 wt % sodium chloride, 0.5 wt % magnesium stearate, and 0.1 wt % Blue Lake #2. The tablet cores were prepared using 13/32-inch standard round concave (SRC) plain-faced tooling. The coating solution contained CA 398-10, PEG 3350, water, and acetone in a weight ratio of 8/2/5/85 (wt %), and amounted to 11.9 wt % of the core weight. Each tablet of DF-5 contained 20 mgA of ziprasidone.

### Dosage Form DF-6

Dosage Form DF-6 was prepared using a co-lyophile of ziprasidone mesylate and sulfobutylether cyclodextrin (SBECD) in the drug-containing composition. The co-lyophile was prepared by freezing an aqueous solution containing SBECD and ziprasidone mesylate in a ratio of 14.7:1 (w/w) and removing the water from the solid state under vacuum. The resulting solid lyophilized cake was milled using a Fitzpatrick M5A mill fitted with a 0.0315-inch rasping plate and a bar impeller.

Dosage Form DF-6 was prepared using the same procedure outlined for DF-1 with the following exceptions. The drug-containing composition consisted of 38.4 wt % of the co-lyophile described above, 60.2 wt % PEO (Polyox WSR N80), 0.4 wt % hydroxypropyl methylcellulose acetate succinate (MF grade from Shin Etsu), and 1 wt % magnesium stearate. The water-swellable composition consisted of 65.0 wt % PEO (Polyox WSR Coagulant), 34.4 wt % sodium chloride, 0.5 wt % magnesium stearate, and 0.1 wt % Blue Lake #2. The tablet cores were prepared using 7/16-inch standard round concave (SRC) plain-faced tooling. The coating solution contained CA 398-10, PEG 3350, water, and acetone in a weight ratio of 7/3/5/85 (wt %), and amounted to 19.5 wt % of the core weight. Each tablet of DF-6 contained 20 mgA of ziprasidone.

### Dosage Form DF-7

Dosage Form DF-7 was prepared using the same procedure outlined for DF-3 with the following exceptions. The drug-containing composition consisted of 10.0 wt % ziprasidone hydrochloride monohydrate, 15.0 wt % HPMCAS (HF grade from Shin Etsu), 74.0 wt % PEO (Polyox WSR N80), and 1.0 wt % magnesium stearate. The drug-containing composition was made by blending the ziprasidone, HPMCAS, and PEO in a Turbula mixer for 20 minutes, passing the blend through a 20-mesh screen, blending an additional 20 minutes, adding the magnesium stearate and blending an additional 4 minutes. The water-swellable composition consisted of 65.0 wt % PEO (Polyox WSR Coagulant), 34.3 wt % sodium chloride, 0.5 wt % magnesium stearate, and 0.2 wt % Blue Lake #2 and was made as outlined for DF-3. The tablet cores were prepared using 15/32-inch SRC tooling. The coating solution contained CA 398-10, PEG 3350, water, and acetone in a weight ratio of 3.5/1.5/3/92 (wt %), and amounted to 18.4 wt % of the core weight. One 900 μm diameter hole was laser-drilled in the coating on the drug-containing composition side of each of the tablets. The resulting bi-layer tablets contained a total of 40 mg active ziprasidone.

### Dosage Form DF-8

Dosage Form DF-8 was prepared using crystals of ziprasidone hydrochloride monohydrate that had been coated with the “H” grade of HPMCAS (HPMCAS-HF, Shin Etsu (where “F” indicates fine)), as previously described. The coated crystals contained 35 wt % active (wt % A) ziprasidone. Dosage Form DF-8 was prepared using the same procedure outlined for DF-1 with the following exceptions. The drug-containing composition consisted of 25 wt % of the coated crystals, 74 wt % PEO (Polyox WSR N80), and 1 wt % magnesium stearate. The water-swellable composition consisted of 65.0 wt % PEO (Polyox WSR Coagulant), 34.3 wt % sodium chloride, 0.5 wt % magnesium stearate, and 0.2 wt % Blue Lake #2. The tablet cores were prepared using 7/16-inch standard round concave (SRC) plain-faced tooling. The coating solution contained CA 398-10, PEG 3350, water, and acetone in a weight ratio of 6.8/1.2/4/88 (wt %), and amounted to 8.1 wt % of the core weight. Each tablet of DF-8 contained 40 mgA of ziprasidone.

### Dosage Form DF-9

Dosage Form DF-9 was prepared using the same procedure outlined for DF-8 except that the coating amounted to 10 wt % of the core weight. Each tablet of DF-9 contained 40 mgA of ziprasidone.

### Dosage Form DF-10

Dosage Form DF-10 consisted of a bilayer osmotic tablet containing coated crystals of ziprasidone hydrochloride monohydrate, that were jet-milled prior to coating to reduce particle size. Dosage form DF-10 was prepared using the following procedures.

### Preparation of Coated Crystals by Spray-Drying

Jet-milled ziprasidone coated crystals were formed by spray drying, as previously described, except that the ziprasidone hydrochloride was first jet-milled to reduce particle size. Jet-milled ziprasidone was prepared by slowly pouring the ziprasidone dry powder into a Glen Mills Laboratory Jet Mill, with two nitrogen lines set at about 100 psi. Milled material was collected in a receiving jar, with a mean particle size of about 2 μm. Jet-milled ziprasidone crystals were coated with HPMCAS-HG, and the properties of the coated crystals after secondary drying were as follows:

| Parameter | Value |
|---|---|
| Morphology | Spherical and wrinkled particles |
| Mean particle diameter (μm) | 44 |
| *Dv₁₀, Dv₅₀, Dv₉₀ (μm) | 13, 40, 81 |
| Span (D₉₀ − D₁₀)/D₅₀ | 1.7 |
| Bulk specific volume (cc/g) | 4.14 |
| Tapped specific volume (cc/g) | 2.65 |
| Hausner ratio | 1.56 |

*10 vol % of the particles have a diameter that is smaller than D₁₀; 50 vol % of the particles have a diameter that is smaller than D₅₀, and 90 vol % of the particles have a diameter that is smaller than D₉₀.

### Preparation of Tablet Cores

The drug-containing composition was prepared using the procedures outlined for DF-7 and consisted of 25.0 wt % ziprasidone coated crystals, 74.0 wt % PEO (Polyox WSR N80), and 1.0 wt % magnesium stearate. The water-swellable composition consisted of 65.0 wt % PEO (Polyox WSR Coagulant), 34.3 wt % sodium chloride, 0.5 wt % magnesium stearate, and 0.2 wt % Blue Lake #2 and was made as outlined for DF-3. The tablet cores were prepared using 7/16-inch SRC tooling. The coating solution contained CA 398-10, PEG 3350, water, and acetone in a weight ratio of 4.25/0.75/2.5/92.5 (wt %), and amounted to 7.8 wt % of the core weight. One 900 μm diameter hole was laser-drilled in the coating on the drug-containing composition side of each of the tablets. The resulting bi-layer tablets contained a total of 40 mg active ziprasidone.

### Dosage Form DF-11

Dosage Form DF-11 was prepared using the same procedure outlined for DF-10 except that the coating amounted to 10.2 wt % of the core weight. Each tablet of DF-11 contained 40 mgA of ziprasidone.

### Dosage Form DF-12

Dosage Form DF-12 consisted of a matrix sustained-release tablet made using coated crystals of ziprasidone hydrochloride. The coated crystals were made using the process previously described, and contained 35 wt % of active ziprasidone coated with HPMCAS-HF. The matrix tablets consisted of 42 wt % of the coated crystals, 42 wt % sorbitol, 15 wt % HPMC (K100LV), and 1 wt % magnesium stearate. The tablets were prepared by first blending the coated crystals, sorbitol, and HPMC in a twin-shell blender for 20 minutes, milling using a Fitzpatric M5A mill, and then blending in the twin-shell blender for an additional 20 minutes. The magnesium stearate was then added and the mixture blended again for 5 minutes. The tablets were manufactured using an F press using 555.5 mg of the mixture using 11-mm SRC plain-faced tooling. The tablet cores were compressed to a hardness of about 11 kp. The resulting sustained-release matrix tablet contained a total of 80 mg active ziprasidone.

### Dosage Form DF-13

Dosage Form DF-13 consisted of a matrix sustained-release tablet made using a mixture of ziprasidone hydrochloride and HPMCAS (HF grade, Shin Etsu) that had been wet granulated. To form the wet granulation, ziprasidone hydrochloride and HPMCAS were mixed in a Turbula mixer for 4 minutes. The resulting physical mixture contained 34 wt % A ziprasidone. A binder solution was then prepared consisting of 10 wt % HPMCAS (HF grade, Shin Etsu) dissolved in an 85/15 (w/w) mixture of isopropyl alcohol/water A 10-gm sample of the physical mixture and a 4-gm sample of the binder solution were then combined in a mortar and pestle and wet granulated by hand. The resulting granules were then dried in a 40° C. oven overnight. The resulting wet granulation contained 36 wt % A ziprasidone.

The matrix tablets consisted of 40 wt % of the wet granulated mixture of ziprasidone hydrochloride and HPMCAS, 44 wt % sorbitol, 15 wt % HPMC (K100LV), and 1 wt % magnesium stearate. The tablets were prepared by first blending the granulated mixture, sorbitol, and HPMC in a twin-shell blender for 20 minutes, milling using a Fitzpatric M5A mill, and then blending in the twin-shell blender for an additional 20 minutes. The magnesium stearate was then added and the mixture blended again for 5 minutes. The tablets were manufactured using an F press using 555.5 mg of the mixture using 11-mm SRC plain-faced tooling. The tablet cores were compressed to a hardness of about 8 kp. The resulting sustained-release matrix tablet contained a total of 80 mg active ziprasidone.

### Dosage Form DF-14

Dosage Form DF-14 consisted of a matrix sustained-release tablet made using coated crystals of ziprasidone hydrochloride. The coated crystals were made using the process previously described, and contained 35 wt % of active ziprasidone coated with HPMCAS (HF grade). The matrix tablets consisted of 30 wt % of the coated crystals, 29 wt % spray-dried lactose, 40 wt % PEO (Polyox WSRN-10) (100,000 daltons), and 1 wt % magnesium stearate. The tablets were prepared by first blending the coated crystals, lactose, and PEO in a twin-shell blender for 20 minutes, milling using a Fitzpatric M5A mill, and then blending in the twin-shell blender for an additional 20 minutes. The magnesium stearate was then added and the mixture blended again for 5 minutes. The tablets were manufactured using an F press using 381 mg of the mixture using caplet-shaped tooling with dimensions 0.30 inches by 0.60 inches. The tablet cores were compressed to a hardness of about 13 kp. The resulting sustained-release matrix tablet contained a total of 40 mg active ziprasidone.

### Dosage Form DF-15

Dosage Form DF-15 consisted of Dosage Form DF-14 that had been coated with an enteric coating. The coating solution consisted of 41.7 wt % Eudragit L30-D55 and 2.5 wt % triethylcitrate in 55.8 wt % water. Coatings were applied in an LDCS-20 pan coater. The coating weight was 10 wt % of the uncoated core weight. The resulting sustained-release matrix tablet contained at total of 40 mg active ziprasidone.

### Dosage Form DF-16

Dosage Form DF-16 consisted of a bi-layer osmotic tablet prepared using the procedures outlined for DF-3 with the following exceptions. The drug layer contained crystals of the tosylate salt form of ziprasidone coated with HPMCAS (H grade) using the procedures outlined for coating crystals of the hydrochloride salt of ziprasidone. The coated crystals contained 35 wt % active ziprasidone. The drug layer composition consisted of 25 wt % of the coated crystals of ziprasidone tosylate, 74 wt % of PEO (Polyox WSR N80), and 1 wt % magnesium stearate. The water-swellable composition consisted of 65.0 wt % PEO (Polyox WSR Coagulant), 34.3 wt % sodium chloride, 0.5 wt % magnesium stearate, and 0.2 wt % Blue Lake #2. The tablet cores were prepared using 7/16-inch standard round concave (SRC) plain-faced tooling. The coating solution contained CA 398-10, PEG 3350, water, and acetone in a weight ratio of 4.25/0.75/2.5/92.5 (wt %), and amounted to 10.4 wt % of the core weight. Each tablet of DF-16 contained 40 mgA of ziprasidone.

### Dosage Form DF-17

Dosage Form DF-17 consisted of a single-layer osmotic tablet that provided sustained release of ziprasidone. The dosage form contained the ziprasidone hydrochloride monohydrate crystals coated with HPMCAS (H grade) as previously described. The tablet core consisted of 26.5 wt % of the coated crystals of ziprasidone, 60.0 wt % sorbitol, 8.0 wt % hydroxy ethyl cellulose (Natrosol 250HX), 1.5 wt % sodium lauryl sulfate (SLS), 3.0 wt % hydroxypropyl cellulose (Klucel EXF), and 1.0 wt % magnesium stearate. To form the tablet core, all of the ingredients except for the magnesium stearate were blended in a twin-shell blender for 15 minutes. The blend was then passed through a Fitzmill M5A equipped with a 0.031-inch Conidur rasping screen at 200 rpm. The blend was then returned to the twin-shell blender and blended an additional 15 minutes. One half of the magnesium stearate was then added to the blend and the mixture blended for an additional 3 minutes. The dry blend was then roller compacted using a Vector Feund TF Mini roller compactor with “S” rolls, using a roll pressure of 390 to 400 psi, a roller speed of 3-4 rpm, and a screw speed of 4-6 rpm. The roller compacted ribbons were then milled using the Fitzmill M5A. The milled material was then returned to a twin-shell blender and blended for 10 minutes, at which time the remaining magnesium stearate was added and the mixture blended for an additional 3 minutes. The tablet cores were then formed using a Killian T100 tablet press using 0.2838-inch by 0.5678-inch modified oval tooling. A coating was applied to the tablet core using the procedures outlined for DF-1, except that the coating solution contained CA 398-10, PEG 3350, water, and acetone in a weight ratio of 4.5/1.5/5/89 (wt %), and amounted to 7.5 wt % of the core weight. Each tablet of DF-17 contained 40 mgA of ziprasidone.

### Dosage Form DF-18

Dosage Form DF-18 consisted of sustained-release multiparticulates prepared using the following procedure. The multiparticulates consisted of 40 wt % ziprasidone hydrochloride monohydrate, 50 wt % COMPRITOL 888 ATO (a mixture of 13 to 21 wt % glyceryl monobehenate, 40 to 60 wt % glyceryl dibehenate, and 21 to 35 wt % glyceryl tribehenate from Gattefossè Corporation of Paramus, N.J.), and 10 wt % poloxamer 407 (sold as LUTROL F127 by BASF Corporation of Mt. Olive, N.J.), and were prepared using the following melt-congeal procedure. First, the COMPRITOL 888 ATO and LUTROL F127 were melted at 90° C. in a heated syringe barrel. The ziprasidone was then added and the suspension of drug in the molten components was stirred for 5 minutes at 700 rpm.

Using a syringe pump, the feed suspension was then pumped at a rate of 75 g/min to the center of a spinning-disk atomizer. The spinning disk atomizer, which was custom made, consisted of a bowl-shaped stainless steel disk of 10.1 cm (4 inches) in diameter. The surface of the spinning disk atomizer was maintained at 100° C. using a thin film heater beneath the disk surface, and the disk was rotated at 10,000 rpm. The multiparticulates formed by the spinning-disk atomizer were congealed in ambient air and a total of 25 g of multiparticulates collected. The average diameter of the smooth, spherical multiparticulates was about 110 μm, as determined by scanning-electron microscopy (SEM).

### Dosage Form DF-19

Dosage Form DF-19 is prepared as follows. First, an enteric coated sustained release core was prepared comprising a matrix sustained-release core containing polymer coated crystals of ziprasidone hydrochloride. The coated crystals were made using the process previously described, and contained 35 wt % of active ziprasidone coated with HPMCAS (H grade). The matrix tablets consisted of 30 wt % of the coated crystals, 29 wt % spray-dried lactose, 40 wt % PEO (Polyox WSRN-10) (100,000 daltons), and 1 wt % magnesium stearate. The tablets were prepared by first blending the coated crystals, lactose, and PEO in a twin-shell blender for 20 minutes, milling using a Fitzpatric M5A mill, and then blending in the twin-shell blender for an additional 20 minutes. The magnesium stearate was then added and the mixture blended again for 5 minutes. The tablets were manufactured using an F press using 381 mg of the mixture using caplet-shaped tooling with dimensions 0.30 inches by 0.60 inches. The tablet cores were compressed to a hardness of about 12-14 kp. The resulting sustained-release matrix tablet contained a total of 40 mg active ziprasidone and had a total mass of about 380 mg.

DF-19 was then coated with an enteric coating. The coating solution consisted of 41.7 wt % Eudragit L30-D55 and 2.5 wt % triethylcitrate in 55.8 wt % water. Coatings were applied in an LDCS-20 pan coater. The coating weight was 10 wt % of the uncoated core weight. The resulting enteric coated sustained-release matrix tablet had a total mass of about 419 mg.

Next, an immediate release coating is applied to the enteric sustained release core. A coating suspension is formed in acetone containing jet-milled ziprasidone and hydroxypropyl methyl cellulose. The drug and polymer collectively are 2 to 15 wt % of the suspension. The suspension is stirred for one hour and is filtered through a 250 μm screen prior to use to remove any particles of polymer that could potentially plug the spray nozzle. The enteric coated sustained release cores are coated in a pan coater. At the conclusion of the spray, the coated dosage forms are dried in a tray drier for one hour at 40° C.

### Dosage Form DF-20

Dosage Form DF-20 is prepared using the same procedure outlined for DF-6 with the following exceptions. The drug-containing composition consists of 38.4 wt % of the co-lyophile described above, 56.1 wt % PEO (Polyox WSR N80), 4.5 wt % hydroxypropyl methylcellulose acetate succinate (HF grade from Shin Etsu), and 1 wt % magnesium stearate.

### Dosage Form DF-21

Dosage Form DF-21 is prepared using the same procedure outlined for DF-6 with the following exceptions. The drug-containing composition consisted of 38.4 wt % of the co-lyophile described above, 56.1 wt % PEO (Polyox WSR N80), 2.25 wt % hydroxypropyl methylcellulose acetate succinate (HF grade from Shin Etsu), 2.25 wt % hydroxypropyl methylcellulose acetate succinate (MF grade from Shin Etsu), and 1 wt % magnesium stearate.

### Dosage Form DF-22

Dosage Form DF-22 is prepared using the same procedure outlined for DF-6 with the following exceptions. The drug-containing composition consists of 38.4 wt % of the co-lyophile described above, 58.4 wt % PEO (Polyox WSR N80), 1.1 wt % hydroxypropyl methylcellulose acetate succinate (HF grade from Shin Etsu), 1.1 wt % hydroxypropyl methylcellulose acetate succinate (MF grade from Shin Etsu), and 1 wt % magnesium stearate.

### Dosage Form DF-23

Dosage Form DF-23 is prepared using the same procedure outlined for DF-14 with the following exceptions. The coated crystals are made using the process previously described, and contained 35 wt % of active ziprasidone coated with a 1:1 mixture of HPMCAS (H grade) and HPMCAS (M grade).

### Dosage Form DF-24

Dosage Form DF-24 consists of Dosage Form DF-23 that are coated with an enteric coating as applied to DF-15. The coated crystals are made using the process previously described, and contained 35 wt % of active ziprasidone coated with a 1:1 mixture of HPMCAS (H grade) and HPMCAS (M grade).

### Dosage Form DF-25

Dosage Form DF-25 is prepared using the same procedure outlined for DF-14 with the following exceptions. The matrix tablet consists of 26.9 wt % of the co-lyophile, 1.65 wt % HPMCAS (H grade, Shin Etsu), 1.65 wt % HPMCAS (M grade, Shin Etsu), 29 wt % spray-dried lactose, 40 wt % PEO (Polyox WSRN-10) (100,000 daltons), and 1 wt % magnesium stearate. The resulting sustained-release matrix tablet contains a total of 20 mg active ziprasidone.

### Control Dosage Form C1

Control dosage form C1 consisted of a commercial GEODON™ capsule containing 40 mgA ziprasidone. The capsule contained ziprasidone hydrochloride monohydrate, lactose, pregelatinized starch, and magnesium stearate.

### Control Dosage Form C2

Control dosage form C2 consisted of 22.65 wt % ziprasidone mesylate trihydrate, 66.10 wt % lactose, 10 wt % pregelatinized starch, and 1.25 wt % magnesium stearate in an immediate release capsule. Each capsule contained 20 mgA of ziprasidone.

### Control Dosage Form C3

Control dosage form C3 consisted of a commercial GEODON™ capsule containing 20 mgA ziprasidone. The capsule contained ziprasidone hydrochloride monohydrate, lactose, pregelatinized starch, and magnesium stearate.

### Control Dosage Form C4

Control dosage form C4 consisted of immediate release tablets containing 20 mgA ziprasidone hydrochloride monohydrate. To form the tablets, 22.61 wt % ziprasidone hydrochloride monohydrate, 51.14 wt % anhydrous lactose, 20.0 wt % microcrystalline cellulose, and 5.0 wt % hydroxypropyl cellulose were initially blended for 30 minutes using a V-blender. Next, 0.75 wt % magnesium was added and blended for 3 minutes. The blend was roller-compacted into ribbons using a Freund TF-mini roller compactor with “DPS” rolls, a rotation speed of 5 rpm, a compaction force of 30 kg/cm², and an auger speed of 18 rpm. The resulting ribbons were granulated using a Comil (197S) fitted with a 2A-1601-173 impeller and a 2A-040G03122329 screen operated at 500 rpm. The granulation had untapped and tapped specific volumes of 1.66 and 1.12 cm³/g, respectively.

The granulated material was added to a twin shell blender and the mixture was blended for 10 minutes. The final amount of magnesium stearate (0.5 wt %) was added and the granulation was blended an additional 3 minutes. A Killian T-100 rotary tablet press with 7/32″ standard round concave (SRC) tooling was used to make 100 mg tablets with a target hardness of 6-8 kiloponds (kP). A White Opadry II film coat (4 wt % of tablet weight) and a Clear Opadry overcoat (0.5 wt % of tablet weight) were applied to the tablets in a Vector/Freund HCT-30 pan coater.

### In Vitro Release Tests

In vitro release tests of DF-1 to DF-18 were performed using direct drug analysis as follows. A dosage form was first placed into a stirred USP type 2 dissoette flask containing 900 mL of a dissolution medium of a simulated intestinal buffer solution. For DF-1 to DF-9, the simulated intestinal buffer consisted of 50 mM NaH₂PO₄ and 2 wt % sodium lauryl sulfate, adjusted to pH 7.5. For DF-10 to DF-13, and DF-16 to DF-18, the simulated intestinal buffer consisted of 50 mM NaH₂PO₄ and 2 wt % sodium lauryl sulfate, adjusted to pH 6.5. For DF-14 and DF-15, the simulated intestinal buffer consisted of 6 mM NaH₂PO₄, 150 mM NaCl, and 2 wt % sodium lauryl sulfate, adjusted to pH 6.5. In the flasks, the dosage form was placed in a wire support to keep the dosage form off of the bottom of the flask, so that all surfaces were exposed to the moving buffer solution and the solutions were stirred using paddles at a rate of 50 or 75 rpm. Samples of the dissolution medium are taken at periodic intervals using a VanKel VK8000 autosampling dissoette with automatic receptor solution replacement. The concentration of dissolved drug in the dissolution medium is then determined by HPLC at a UV absorbance of 315 nm using a Zorbax RxC8 Reliance column and a mobile phase consisting of 55% (50 mM potassium dihydrogen phosphate, pH 6.5)/45% acetonitrile. Drug concentration was calculated by comparing UV absorbance of samples to the absorbance of drug standards. The mass of dissolved drug in the dissolution medium was then calculated from the concentration of drug in the medium and the volume of the medium, and expressed as a percentage of the mass of drug originally present in the dosage form. Results are shown in Table 6.

TABLE 6

| Time | Ziprasidone Released (wt %) | | | | | | | | |
|---|---|---|---|---|---|---|---|---|---|
| (hrs) | DF-1 | DF-2 | DF-3 | DF-4 | DF-5 | DF-6 | DF-7 | DF-8 | DF-9 |
| 0 | 0 | 0 | 0 | 0 | 0 | 0 | 0 | 0 | 0 |
| 1 | — | — | 0 | 0 | 5 | — | 0 | — | — |
| 2 | 16 | 4 | 18 | — | 20 | 6 | 12 | — | — |
| 3 | — | — | — | — | — | — | — | 9 | 7 |
| 4 | 43 | 19 | 44 | — | 52 | 26 | 40 | — | — |
| 5 | — | — | — | 16 | — | — | — | — | — |
| 6 | — | — | 72 | — | — | — | 68 | 27 | 20 |
| 8 | 75 | 47 | — | 45 | 72 | 65 | — | — | — |
| 9 | — | — | 99 | — | — | — | 98 | — | — |
| 10 | 86 | 65 | — | 61 | — | — | — | — | — |
| 12 | 89 | 77 | 99 | 75 | 88 | 90 | 99 | 71 | 59 |
| 14 | 91 | 87 | 100 | — | — | — | 99 | — | — |
| 15 | — | — | — | — | — | — | — | — | — |
| 16 | 92 | 92 | 99 | 88 | 94 | 98 | 99 | 92 | 81 |
| 18 | — | — | 99 | — | — | — | 98 | — | — |
| 20 | — | — | 98 | — | — | — | 98 | — | — |
| 24 | 91 | 94 | — | 91 | 98 | 96 | — | 96 | 91 |

| Time | Ziprasidone Released (wt %) | | | | | | | | |
|---|---|---|---|---|---|---|---|---|---|
| (hrs) | DF-10 | DF-11 | DF-12 | DF-13 | DF-14 | DF-15 | DF-16 | DF-17 | C1 |
| 0 | 0 | 0 | 0 | 0 | 0 | 0 | 0 | 0 | 0 |
| 1 | 0 | 0 | 7 | 9 | 17 | 4 | 0 | 1 | 95 |
| 2 | 4 | 1 | — | — | 38 | 22 | 1 | — | 98 |
| 3 | — | — | 27 | 29 | 60 | 43 | — | 13 | |
| 4 | 21 | 13 | — | — | 79 | 65 | 14 | — | |
| 5 | — | — | 47 | 54 | 95 | 83 | — | — | |
| 6 | 37 | 27 | — | — | 100 | 96 | 29 | 42 | |
| 8 | — | — | — | — | 100 | 100 | — | 57 | |
| 9 | 66 | 49 | — | — | — | — | 53 | — | |
| 10 | — | — | 88 | 90 | 100 | 100 | — | — | |
| 12 | 92 | 72 | — | — | — | — | 76 | 73 | |
| 14 | 97 | 85 | — | — | — | — | 89 | — | |
| 15 | — | — | 100 | 101 | — | — | — | — | |
| 16 | 97 | 96 | — | — | — | — | 96 | 82 | |
| 18 | 99 | 101 | — | — | — | — | 96 | — | |
| 20 | 99 | 100 | 100 | 100 | — | — | 96 | 86 | |
| 24 | — | — | — | — | — | — | — | 88 | |

The results for the immediate release (IR) commercial GEODON™ capsule showed that more than 95 wt % of the ziprasidone had been released during the first 2 hours after introduction to the in vitro test media.

In vitro tests of multiparticulate dosage form DF-18 were performed using the direct drug analysis method described above with the following exceptions. The multiparticulate dosage form was placed into a small beaker and pre-wet with a sample of the dissolution medium. The pre-wetted multiparticulates were then added to the dissolution medium at time zero. The dissolution medium was stirred using paddles at a rate of 50 rpm. A sufficient amount of the multiparticulates were added to the dissolution medium so that the concentration of ziprasidone, once all of the ziprasidone was released, was 90 μgA/mL. Drug concentrations were determined using HPLC as described above. The results are in Table 7.

TABLE 7

| | DF-18 |
|---|---|
| Time (hrs) | Ziprasidone Released (wt %) |
| 0 | 0 |
| 0.5 | 17 |
| 1 | 29 |
| 2 | — |
| 3 | 60 |
| 4 | — |
| 5 | 78 |
| 6 | — |
| 8 | — |
| 10 | — |
| 15 | — |

From the data in Tables 6 and 7, the times to release 80 wt % and 90 wt % of the ziprasidone originally present in the dosage forms were estimated and are provided in Table 8.

TABLE 8

| | Approximate Time to | Approximate Time to |
|---|---|---|
| Dosage | Release 80 wt % of | Release 90 wt % of |
| Form | the Ziprasidone (hr) | the Ziprasidone (hr) |
| DF-1 | 9 | 13 |
| DF-2 | 13 | 15 |
| DF-3 | 7 | 8 |
| DF-4 | 14 | 20 |
| DF-5 | 10 | 14 |
| DF-6 | 10 | 12 |
| DF-7 | 7 | 8 |
| DF-8 | 14 | 16 |
| DF-9 | 16 | 24 |
| DF-10 | 11 | 12 |
| DF-11 | 13 | 15 |
| DF-12 | 8 | 11 |
| DF-13 | 8 | 10 |
| DF-14 | 4 | 5 |
| DF-15 | 5 | 6 |
| DF-16 | 13 | 14 |
| DF-17 | 15 | >24 |
| DF-18 | 5 | >5 |
| C1 | <1 | <1 |

### Example 1

The sustained release Dosage Forms DF-1 and DF-2 and the Control Dosage Form C1 were tested in in vivo tests in humans in a Phase 1, Open, Randomized, Crossover, Single-Dose study in healthy subjects. Healthy human volunteers were dosed with the dosage forms in the fed state, each dosage form containing 40 mgA ziprasidone.

Plasma samples were collected at multiple times post-dose and ziprasidone concentrations were determined. Table 9 shows Cₘₐₓ (ng/mL), AUC_{0-inf} (ng-hr/mL), and Tₘₐₓ (hr) obtained for these tests. The results provided in Table 9 are after the initial dose and are not steady state values.

TABLE 9

| | Cₘₐₓ | AUC_{0-inf} | Tₘₐₓ | C₁₂ | C₂₄ | Cₘₐₓ/ |
|---|---|---|---|---|---|---|
| Dosage Form | (ng/mL) | (ng-hr/mL) | (hr) | (ng/mL) | (ng/mL) | C₂₄ |
| DF-1 | 99 | 887 | 6 | 44 | 8 | 12.0 |
| | (30) | (266) | | | | |
| DF-2 | 52 | 701 | 9 | 38 | 12 | 3.8 |
| | (16) | (337) | | | | |
| C1 (40 mgA | 117 | 1006 | 6 | 39 | 7 | 15.1 |
| commercial | (45) | (290) | | | | |
| IR capsule) | | | | | | |

The data in Table 9 show that the sustained-release dosage forms DF-1 and DF-2 provided Cₘₐₓ values that were lower than that of the IR control, providing Cₘₐₓ values that were 85% and 44% that provided by C1, respectively. Furthermore, the ratio of Cₘₐₓ/C₂₄ for DF-1 and DF-2 were lower than that provided by C1.

### Example 2

The sustained-release dosage forms DF-4 and DF-5 were tested in in vivo tests in humans using the procedures outlined in Example 1. Healthy human volunteers were dosed with the dosage forms in the fed state. Each subject was dosed two tablets of DF-5 so that 40 mgA of ziprasidone was dosed.

Plasma samples were collected at multiple times post-dose and ziprasidone concentrations were determined. Table 10 shows Cₘₐₓ(ng/mL), AUC_{0-inf} (ng-hr/mL), and Tₘₐₓ (hr) obtained for these tests, as well as C₁₂ and C₂₄ values. The results provided in Table 10 are after the initial dose and are not steady state values. Also included in Table 10 are the results for the IR Control C1, previously described.

TABLE 10

| | Cₘₐₓ | AUC_{0-inf} | Tₘₐₓ | C₁₂ | C₂₄ | Cₘₐₓ/ |
|---|---|---|---|---|---|---|
| Dosage Form | (ng/mL) | (ng-hr/mL) | (hr) | (ng/mL) | (ng/mL) | C₂₄ |
| DF-4 | 38.8 ± 14.4 | 439 ± 176 | 8.3 ± 2.9 | 26.9 ± 21.3 | 5.3 ± 2.6 | 7.3 |
| DF-5 | 39.0 ± 10.1 | 458 ± 138 | 7.6 ± 1.8 | 25.8 ± 13.1 | 5.7 ± 1.9 | 6.8 |
| (2 tablets) | | | | | | |
| C1 (40 mgA | 106 | 1009 | 6 | 39 | 7 | 15.1 |
| commercial | | | | | | |
| IR capsule) | | | | | | |

The data in Table 10 show that the sustained-release dosage forms DF-4 and DF-5 provided Cₘₐₓ values that were lower than that of control C1, providing Cₘₐₓ values that were 37% that provided by C1, respectively. Furthermore, the ratios of Cₘₐₓ/C₂₄ for DF-4 and DF-5 were lower than that provided by C1.

### Example 3

The sustained release dosage forms DF-3, DF-7, DF-8, DF-9, DF-10, DF-11, DF-15, and control dosage form C1 were tested in in vivo tests using beagle dogs in the fed state. The dogs were fed one can of Clinicare Canine Liquid Diet the day before the study. Dogs were allowed ad libidum access to water. On the morning of the study, dogs were fed 50 g of dry food and allowed 15 minutes to eat. After the dogs finished eating, the dosage form specified was administered with 50 mL of water via gavage immediately after dose administration. Dogs were then placed in metabolism cages or individual runs for the duration of the study. They were allowed free access to water and fed normal rations 8 hours after dose administration.

Whole blood samples of 6 ml were taken from the jugular or cephalic vein using a plasma serum separator tube containing sodium heparin with a 20 gauge needle at 0, 0.5, 1, 2, 4, 8, 12, and 24 hours post dosing. Samples were spun in a refrigerated (5° C.) centrifuge at 2500 rpm for 15 minutes. The resultant plasma samples were poured into 2 ml cryogenic plastic tubes and stored in a freezer (−20° C.) within 30-minutes post sampling time. Samples were then analyzed using HPLC. Table 11 summarizes the results of these tests. The results provided in Table 11 are after the initial dose and are not steady state values.

TABLE 11

| | Cₘₐₓ | AUC_{0-inf} | Tₘₐₓ | C₁₂ | C₂₄ | Cₘₐₓ/ |
|---|---|---|---|---|---|---|
| Dosage Form | (ng/mL) | (ng-hr/mL) | (hr) | (ng/mL) | (ng/mL) | C₂₄ |
| DF-3 | 112 ± 26 | 877 ± 202 | 8 | 46.1 ± 19.5 | 3.0 ± 0.6 | 37.3 |
| (40 mgA) | | | | | | |
| DF-7 | 105 ± 28 | 824 ± 254 | 5.3 | 27.4 ± 8.9 | 3.7 ± 2.4 | 28.4 |
| (40 mgA) | | | | | | |
| DF-8 | 107.5 ± 50.0 | 798 ± 311 | 8 | 38.6 ± 12.5 | 4.9 ± 3.2 | 21.9 |
| (40-mgA) | | | | | | |
| DF-9 | 50.9 ± 28.4 | 381 ± 118 | 7.3 | 19.3 ± 6.8 | 4.3 ± 2.7 | 11.8 |
| (40-mgA) | | | | | | |
| DF-10 | 87 ± 24 | 643 ± 153 | 8 | 32.1 ± 8.4 | 4.8 ± 2.9 | 18.1 |
| (40 mgA) | | | | | | |
| DF-11 | 47 ± 32 | 342 ± 189 | 7.3 | 16.4 ± 10.1 | 3.3 ± 1.2 | 14.2 |
| (40 mgA) | | | | | | |
| DF-15 | 110 ± 48 | 510 ± 210 | 10 | 50.3 ± 19.7 | 7.4 ± 9.2 | 14.9 |
| (40 mgA) | | | | | | |
| Control C1 | 282 ± 122 | 1890 ± 452 | 3.1 | 51.5 ± 20.8 | <3 | >94 |
| (40-mgA | | | | | | |
| IR Capsule) | | | | | | |

The data in Table 11 show that the sustained release dosage forms provided a lower Cₘₐₓ than the IR control C1, with Cₘₐₓ values that were 17% to 40% those obtained with C1. The sustained release dosage forms also provided ratios of Cₘₐₓ/C24 that were significantly lower than that provided by the IR control (C1), with values that ranged from less than 13% to less than 40% of C1.

### Example 4

Studies were conducted in man of both immediate release and sustained release ziprasidone dosage forms, and the results were used as the basis for a modeling study to determine appropriate dosage forms to achieve desired steady state concentrations of ziprasidone in the blood. The modeling results may be used to prepare dosage forms that provide preferred Cₘₐₓ(blood), Cₘᵢₙ (blood), and Cₘₐₓ/Cₘᵢₙ ratios.

Blood concentration versus time data were collected from the results of the study conducted in Example 1 for the sustained release dosage form DF-2 and the IR oral capsule C1. In addition, blood concentration versus time data were collected from a separate study for the immediate release tablet C4. The data were fit using a one compartment pharmacokinetic model with first order absorption and elimination. The mean pharmacokinetic parameters derived from the model are reported in Table 12:

TABLE 12

| | CL/F | V | Kₐ | T_{lag} | AUC |
|---|---|---|---|---|---|
| Formulation | (L/hr) | (L) | (1/hr) | (hr) | (ng-hr/mL) |
| C1 | 43.8 | 282 | 0.44 | 0.95 | 913 |
| | | | | | (1016)* |
| DF-2 | 58.1 | 250 | 0.14 | 2.8 | 690 |
| | | | | | (639)* |
| C4 | 36.4 | 143.4 | 0.37 | 0.46 | 550 |
| | | | | | (558)* |

*Mean AUC from previous NCA analysis
(CL/F = Clearance/Oral Bioavailability; V = volume of distribution; Kₐ = Absorption rate constant ; T_{lag} = time lag; and AUC = concentration of ziprasidone in the blood area under the curve).

The results of the model were then used to calculate various steady state blood concentrations of ziprasidone (plasma) for various model dosage forms at different dosing intervals. The calculated steady state blood (plasma) ziprasidone concentrations and pharmacokinetic parameters are shown in Table 13:

TABLE 13

| | Amount | Dosing | Tₘₐₓ | Cₘₐₓ | Cₘᵢₙ | AUC_{0-τ} | Cₘₐₓ/Cₘᵢₙ |
|---|---|---|---|---|---|---|---|
| Formulation | Drug (mgA) | Interval | (hr) | (ng/mL) | (ng/mL) | (hr*ng/mL) | Ratio |
| C1 | 30 | BID | 4 | 77.6 | 29.5 | 681 | 2.63 |
| C1 | 40 | BID | 4 | 103 | 39.4 | 908 | 2.61 |
| C1 | 60 | BID | 4 | 155 | 59 | 1360 | 2.63 |
| C1 | 120 | QD | 4.61 | 250 | 16.6 | 2750 | 15.1 |
| DF-2 | 30 | BID | 6.79 | 52.1 | 30.6 | 526 | 1.70 |
| DF-2 | 40 | BID | 6.79 | 69.4 | 40.8 | 702 | 1.70 |
| DF-2 | 60 | BID | 6.79 | 104 | 61.2 | 1050 | 1.70 |
| DF-2 | 90 | BID | 6.79 | 156 | 91.8 | 1580 | 1.70 |
| DF-2 | 120 | BID | 6.79 | 208 | 122 | 2110 | 1.70 |
| DF-2 | 120 | QD | 8 | 148 | 25.1 | 2110 | 5.90 |
| C4 | 20 | BID | 3.39 | 69.6 | 17.2 | 549 | 4.05 |
| C4 | 30 | BID | 3.39 | 104 | 25.8 | 824 | 4.03 |
| C4 | 45 | BID | 3.39 | 157 | 38.6 | 1240 | 4.07 |
| C4 | 60 | BID | 3.39 | 209 | 51.5 | 1650 | 4.06 |
| C4 | 60 | QD | 3.64 | 185 | 3.01 | 1650 | 61.5 |

(BID = dosing twice daily; QD = dosing once daily; Tₘₐₓ is time in hours to Cₘₐₓ)

The results show that each of the sustained release dosage forms are predicted to achieve improved performance relative to the IR oral capsule and IR tablet. For example, comparing the 60 mgA IR oral capsule with the 60 mgA sustained release dosage form, the sustained release dosage form significantly lowers Cₘₐₓ, while providing about the same Cₘᵢₙ. The Cₘₐₓ for the 60 mgA IR oral capsule is predicted to be 155 ng/ml, while the Cₘₐₓ for the 60 mg sustained release dosage form is predicted to be 104 ng/ml.

The modeling further indicates that higher doses of ziprasidone may be administered in a sustained release dosage form without increasing Cₘₐₓ relative to an IR dosage form containing the same amount of ziprasidone. For example, the model predicts that a 90 mgA sustained release dosage form will provide a Cₘₐₓ of 156 ng/ml and a Cₘᵢₙ of 91.8 ng/ml. In contrast, an IR oral capsule would provide a Cₘₐₓ of 155 ng/ml, but a Cₘᵢₙ of only 59 ng/ml. Thus, the model predicts that a sustained release dosage form having 50% more ziprasidone does not significantly increase Cₘₐₓ, but does significantly increase Cₘᵢₙ compared with an IR oral capsule.

In addition, the sustained release dosage form provides calculated steady state blood (plasma) ziprasidone concentrations that would permit once a day administration for certain doses of ziprasidone. The sustained release dosage form containing 120 mgA ziprasidone when administered once per day provides a Cₘᵢₙ of 25.1 ng/ml and a Cₘₐₓ of 148 ng/ml, which are both within the scope of the desired steady state blood concentrations for ziprasidone. In contrast, an IR oral capsule containing 120 mgA ziprasidone is predicted to provide a Cₘᵢₙ of 16.6 ng/ml, which is less than the desired minimum ziprasidone blood concentration of 20 ng/ml.

Finally, the results of the model were then combined to predict performance of dosage forms having both immediate release and sustained release portions. The modeling results for DF-2 were combined with the modeling results from C4 by assuming that the dose response was simply linear. For example, the “SR30+IR30” formulation corresponds with a dosage form having a 30 mgA sustained release portion and a 30 mgA immediate release portion, in which the sustained release portion behaves like DF-2, and the immediate release portion behaves like C4. Results of the model are shown in Table 15, with the calculated results for a 60 mgA immediate release oral capsule (C1) shown for comparison:

TABLE 15

| Formulation | Dosing | Tₘₐₓ | Cₘₐₓ | Cₘᵢₙ | AUC_{0-τ} | Cₘₐₓ/Cₘᵢₙ |
|---|---|---|---|---|---|---|
| SRmgA + IRmgA | Interval | (hr) | (ng/mL) | (ng/mL) | (hr*ng/mL) | Ratio |
| SR30 + IR30 | BID | 4.24 | 146 | 63.7 | 1340 | 2.29 |
| SR30 + IR45 | BID | 3.88 | 196 | 76.2 | 1750 | 2.57 |
| SR30 + IR60 | BID | 3.76 | 248 | 88.9 | 2160 | 2.79 |
| SR40 + IR30 | BID | 4.61 | 161 | 76.6 | 1520 | 2.1 |
| SR40 + IR45 | BID | 4.24 | 211 | 88.9 | 1930 | 2.37 |
| SR40 + IR60 | BID | 3.88 | 262 | 102 | 2340 | 2.57 |
| SR60 + IR30 | BID | 4.85 | 193 | 102 | 1870 | 1.89 |
| SR60 + IR45 | BID | 4.36 | 242 | 115 | 2280 | 2.1 |
| SR60 + IR60 | BID | 4.24 | 292 | 128 | 2690 | 2.28 |
| SR90 + IR30 | BID | 5.21 | 242 | 141 | 2400 | 1.72 |
| SR90 + IR45 | BID | 4.85 | 289 | 153 | 2810 | 1.89 |
| SR120 + IR30 | BID | 5.21 | 292 | 179 | 2930 | 1.63 |
| C1 (60 mgA) | BID | 4 | 155 | 59 | 1360 | 2.63 |

(SR corresponds with parameters derived from DF-2, while IR corresponds with parameters derived from C4).

The results show that dosage forms that have both immediate release and sustained release portions are predicted to achieve good performance. All of the dosage forms are predicted to achieve a steady state Cₘᵢₙ of greater than 50 ng/ml, and a Cₘₐₓ of less than 330 ng/ml. Several of the dosage forms are predicted to provide a steady state Cₘᵢₙ that is greater than 50 ng/ml and a steady state Cₘₐₓ that is less than 200 ng/ml: SR30+IR30; SR30+IR45; SR40+IR30; and SR60+IR30.

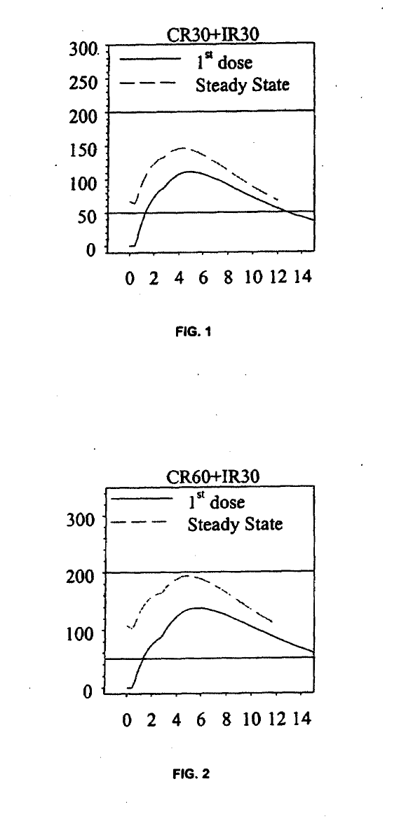

FIG. 1 shows ziprasidone blood concentrations calculated from the model for the SR30+IR30 dosage form. The solid line shows the calculated ziprasidone blood concentration (plasma) after the initial dose, while the dashed line shows the steady state ziprasidone blood concentration (plasma). FIG. 2 shows the calculated results for the SR60+IR30 dosage form. In both cases, dosage forms are predicted to achieve a steady state Cₘᵢₙ of greater than 50 ng/ml, and a steady state Cₘₐₓ of less than 200 ng/ml.

The terms and expressions which have been employed in the foregoing specification are used therein as terms of description and not of limitation, an there is no intention, in the use of such terms and expressions, of excluding equivalents of the features shown and described or portions thereof, it being recognized that the scope of the invention is defined and limited only by the claims which follow.

1-50. (canceled)

51. Ziprasidone nanoparticles having an average crystal size of less than about 500 nm.

52. Ziprasidone nanoparticles having an average crystal size of less than about 250 nm.

53. Ziprasidone nanoparticles having an average crystal size of less than about 100 nm.

54. Ziprasidone nanoparticles according to claim 51 prepared in the presence of a surface modifier.

55. Ziprasidone nanoparticles according to claim 51 contacted with a surface modifier.

56. Ziprasidone nanoparticles according to claim 52 prepared in the presence of a surface modifier.

57. Ziprasidone nanoparticles according to claim 52 contacted with a surface modifier.

58. Ziprasidone nanoparticles according to claim 53 prepared in the presence of a surface modifier.

59. Ziprasidone nanoparticles according to claim 53 contacted with a surface modifier.

60. A sustained release oral dosage form comprising a pharmaceutically effective amount of ziprasidone, said ziprasidone in the form nanoparticles having an effective average crystal size of less than about 500 nm, and a sustained release means for releasing at least a portion of said ziprasidone, wherein following administration to achieve steady state, said dosage form provides a steady state minimum blood ziprasidone concentration (Cₘᵢₙ) of at least 20 ng/ml, and a steady state maximum blood ziprasidone concentration (Cₘₐₓ) of less than 330 ng/ml.

61. A sustained release oral dosage form according to claim 60, said ziprasidone nanoparticles contacted with a surface modifier.

62. A sustained release oral dosage form according to claim 60, said ziprasidone nanoparticles prepared in the presence of a surface modifier.

63. A sustained release oral dosage form according to claim 60, said ziprasidone nanoparticles having an effective average particle size of less than about 250 nm.

64. A sustained release oral dosage form according to claim 63, said ziprasidone nanoparticles having an effective average particle size of less than about 100 nm.

65. A sustained release oral dosage form comprising a pharmaceutically effective amount of ziprasidone, said ziprasidone in the form nanoparticles having an effective average crystal size of less than about 500 nm, and said dosage form releasing no greater than 90 wt % of said ziprasidone from said dosage form during the first 2 hours after administration to an in vitro use environment, wherein said dosage form comprises at least 30 mgA of ziprasidone, and said in vitro use environment is 900 mL of a dissolution medium of a simulated intestinal buffer solution.

66. A sustained release oral dosage form according to claim 65, said ziprasidone nanoparticles contacted with a surface modifier.

67. A sustained release oral dosage form according to claim 65, said ziprasidone nanoparticles prepared in the presence of a surface modifier.

68. A sustained release oral dosage form according to claim 65, said ziprasidone nanoparticles having an effective average particle size of less than about 250 nm.

69. A sustained release oral dosage form according to claim 68, said ziprasidone nanoparticles having an effective average particle size of less than about 100 nm.
